SEQUENCE LISTING (7 sequences)

SEQ ID NO: 1 - protein, 290 aa, Haemophilus parasuis
GGGSSGGHQQAVDKTENPITNQQVSENNPQSLDKNNASKAPEAKEDKQIKEPIVGKQSKEKIEPPSQKLNDQDIWQGFSKNGGYYDASNWVSITLEGKEIKLAPVNGSNSNNSKMNTLRDADGNLVGYYGYATLSKTKPDPYRPDENNVERKYMALQEVTAREEAKTRPTHDLSYQGKMYYALKDAPEQAKEANVTATYKHMNKSISINIYGVNGNDLTTNQTNVSENGYIFTRLYLMGKPKIPAGQFDGGLYGKSGEILLGKAKNEDATPHTPQKNWEGVIGATATKDK

SEQ ID NO: 2 - protein, 329 aa, Haemophilus parasuis
CNKEPQQIAFEGKTMGTTYHIKYIDDGSLTNLPKPEEVQRQMDTLLKMVNNEMSTYQKDSQISQFNQMKEADKPFEIASDFAKVVKEAVRLNKVTEGALDVTVGPLVNLWGFGPDKRLNKAPTEEQIAERATWVGIEKLVLDDNATKATLTKKVPELYLDLSAIAKGFGVDKLADYLEQLGVQHYLVEIGGELRGKGLNLKGQPWRIAVEKPEFAQGTSSQIVVPLHNLGMATSGNYRNYFEDEQGNRLSHIIDPKALRPISHNLASITVFAPTTMTADGLSTGLYVLGAEKALEVAERENLAIFLIIKKGDGYETKMSSAFEKLINQK

SEQ ID NO: 3 - protein, 549 aa, Haemophilus parasuis
CSGNKVTETLKNEAYGNSEFYINKAEQSRNIEEQQSYRLLAVRKLIEENKVVEAQNTFSEILVAKLNDEQKLEHRLLIAQLAALQGNNSEAQGVLRALPQTLLSQSQRLRVYQTQARIAENQNDVIAAVNARALMDRYMTDTQSRQANNDKIWEMLRNANRGMLEKAVAGPGEIGLAGWLALVVAYNQNMSNPAQLPQVIEMWKQQYPNHSAVLLLPIELRNVSSFQQTQLNGVALLLPLSGDAKILGEIIKRGFDDAKGTTTMQVQVFDTDSAPINDLLMQAKQQGAQTIIGPLLKPRVDEMLTSPEISNINVLALNSTPNVRAVAKVCYYGLSPEAEARSAAERFLKDGLQHAVVVAPNGDFGTRSAEAFAQRWRQLTNRDTDIRYYNQAFEVTSLLQGSGIGQGSGLYALGTAEQLSDLKQSLDNSPLANQFPIYTSSRSNSPNNGPDFRLTMEGVKFSEIPLLSDPSSSEYKKAEQIVESDFSMMRLYAMGSDTWSIANKFNEFRQIPGYKVHGLTGVLSAGPNCNIEREMNWLQYRGGSIVDAN

SEQ ID NO: 4 - protein, 783 aa, Haemophilus parasuis
SPFVIKDIRIDGIQTSVSQTMIPTLPVKVGQLATDNDIANVIRHLFVQNRFQNISATREGDILIIKVVENPVINNVDIEGNSAIPKEPLEQNLKANLITKGELFNSTKLDAFKSALIEHYHSIGRYNATIDTVITNSDNGGVNIKLDIKEGDVAYVKAINFEGNQAFTSKVLMKQLDIQPDVSWFNIFQSSKFEQTAYNQDLENLRNFYMNQGYAKFNVEAEQPVFSDDKKEVSLTYKINEGIPYTVSEIRIVGNTAKLDNELNEQLKYYKTGELFSKNELSKIEDNIKQVFGNNGFASAKIELHPTFDDTNKTIRLTFVVDAGQRVYVRQIRFEGNDVTADSTLRREMRQQEGAWLSTSLAALGKARLERTGFYDSVEMSYPNVPNTADQVDIVYKIKERNTGSINFGVGYGTESGLSYQAGIKQDNFLGMGSTISLNGTRNDYGTSINLGYTEPYFTKDGVSLGGNIFYEDYDNSKNNSVASYKRQTYGVNSTLGFPVNEYNSYFLGLGYTQDLIKNARREFNRELYVQSMKFPIDNDLNVYKKIKANDFDFSFGWNYNSLNRGFLPTEGTLTSISGRITTPGSDNKYYKVSIDFKNYLPVNREHKWVISTKANIAYANGFSGKELPFYQTYTAGGIGSLRGFAYGAIGPKAIYLGKDNIRFDSVGDIVGGNAMASATLELITPTPFVSEKYQHNVRTSLFVDAATVLNTKWKTNPVVNDIQYNYSSLENFKDYKRFRASTGIAFQWNSPIGPLVFSYAKPIKKYQGDEIEQFQFSIGGSF

SEQ ID NO: 5 - protein, 368 aa, Haemophilus parasuis
QDIDYGIAAPQLNAQTYILMDYNSGAVLASLNPDQRQAPASLTKMMTSYVVGEALKTGKIKNSDLVTVTENSWGQKFPGSSKMFLNVNQQVSVEDLNKGIIVVSGNDACVAIAEHISGSEQTFIDSMNKFVQKFGLKNTHFATVHGLDHPEQYSSARDMAIIGAHIIRDLPEEYKIYSEKEFTYNKITQPNRNGLLWDKSINVDGMKTGHTDQAGYNLVASAYNSNTRLISVVMGVPTYKGREVESKKLLQWGFANFETVKPLSAGQSVLEQAVYYGDLSKAQLGTLQDSYITVPKGKTAELKARYTLEKTNLEAPLVKGQVVGKVIYQLNGKDVASVDLQVMHDVQEAGIFGKAWDWIVLTVKGLFN

SEQ ID NO: 6 - protein, 369 aa, Haemophilus parasuis
EKLEQKPIIELRSITKRYGEKTIIDGLDLTINDGEFLTILGPSGCGKTTVLRLIAGFEDLTDGSIILDGQDVSNVDAEQRPVNTVFQSYALFPHMTIFDNVAFGLRMQKVPEAEIKPRVMEALRMVRLEEMAPRKPSQLSGGQQQRIAIARAVVNQPKVLLLDESLSALDYKLRKEMQHELKQLQRQLGITFIFVTHDQEEALTMSDRIIVMNKGKIQQDGSPREIYEEPSNLFVARFIGEINVFDATVIERVTEKTVKANVEGRICEIHTELPVHPNQKLKVLLRPEDIVIEELDENESAKAVIGHVSDRTYKGMTLESNVVLDHNQMKVLVSEFFNEDDPHMDHSVGQKVALTWHEGWEVVLDDEDC

SEQ ID NO: 7 - protein, 481 aa, Haemophilus parasuis
YKCPFDHGSKTLTTAAGAPVVDNDNTMSAGPRGPLLLQDVWFQEKLAHFARERIPERVVHAKGSAAYGTFTVTADITKYTKAAVFKPGAQTEVLLRFSTVAGERGAADAERDVRGFSLKFYTEQGNWDLVGNNTPVFFIRDPLKFPDFIHTQKRNPQTNLRDANAAWDFWSRHPESMHQIMTLFSDRGIPATLRHMNGYGSHTYSFVNANNERFWVKFHFKTQQGHKFFTNEEAAKVVGEDRESSQKDLYEAIERGEFPRWTVQVQIMPEADAHKHNYAFDLTKVWPHADYPVIEVGVLELNRNPQNYFAEVEQAAFAPSNIVPGIGFSPDRMLQGRLFSYQDAQRYRLGVNHHQIPVNAPKCSYHTTHRDGAMRVDANGGNHPNYAPNRFDTYVPTHDQLPLQIEREAAHFNFREYDEDYYSQPAALYNLFTAEEKDRLAGNFAAGLSGVTIPEIVERQMAHFEKVSPELANAIRQKLAK

(19) United States
(12) Patent Application Publication
(10) Pub. No.: US 2025/0228924 A1
(43) Pub. Date: Jul. 17, 2025

(54) IDENTIFICATION OF G. PARASUIS IMMUNOGENIC PROTEINS AND SUBUNIT VACCINE COMPRISING SUCH

(71) Applicant: The United States of America, as Represented by the Secretary of Agriculture, Washington, DC (US)

(72) Inventors: SAMANTHA J. HAU, ANKENY, IA (US); JARLATH E. NALLY, AMES, IA (US); JOHN D. LIPPOLIS, AMES, IA (US); KIRSTEN C. EBERLE, ELKHORN, NE (US); SUSAN BROCKMEIER, NEVADA, IA (US)

(21) Appl. No.: 18/990,203

(22) Filed: Dec. 20, 2024

Related U.S. Application Data
(60) Provisional application No. 63/620,071, filed on Jan. 11, 2024.

(51) Int. Cl. A61K 39/102; A61K 39/00; A61P 31/04

(52) U.S. Cl. CPC A61K 39/102; A61P 31/04; A61K 2039/521; A61K 2039/552

(57) ABSTRACT

Provided herein are methods to identify immunogenic G. Parasuis proteins, compositions comprising such immunogenic proteins, kits comprising such immunogenic proteins, and methods of using such immunogenic proteins to prevent G. Parasuis in pigs.

### CROSS-REFERENCE TO RELATED APPLICATIONS

This application claims the benefit of U.S. Provisional Patent Application No. 63/620,071, filed Jan. 11, 2024, which is incorporated herein by reference in its entirety.

### TECHNICAL FIELD

The disclosure relates the identification of Glasserella Parasuis immunogenic proteins and subunit vaccines for the field of pharmaceutical biotechnology, specifically to vaccines for G. Parasuis protection and uses thereof.

### SEQUENCE LISTING

The instant application contains a Sequence Listing XML required by 37 C.F.R. § 1.831 (a) which has been submitted in XML file format via the USPTO patent electronic filing system, and is hereby incorporated by reference in its entirety. The XML file was created on Jan. 11, 2024, is named Sequence_Listing_0046_23, and has 1000 bytes.

### BACKGROUND

Glaesserella (Haemophilus) parasuis, the causative agent of Glasser's disease in swine, is a gram-negative bacteria of the family Pasteurellaceae. Disease with G. parasuis is characterized by arthritis, polyserositis, and meningitis with clinical signs including swollen joints and lameness, labored breathing, and seizures. The 15 serovars of G. parasuis were initially defined using serology, but more recently, genetic serotyping via PCR has been implemented, which assesses the composition of the capsular locus. A range of virulence exists among, and within, the 15 serovars. Strain variation has made developing broadly protective immunity difficult and often bacterin vaccines result in serotype or strain specific immunity.

Because of the difficulty generating broadly-effective bacterin vaccines against G. parasuis, more recent vaccine work has focused on alternative platforms, such as subunit vaccines. Subunit vaccines employ purified proteins from the bacterium to direct the immune response to highly conserved, immunogenic, surface exposed proteins that are important to protection. Identification of subunit candidates has been accomplished thus far using hyperimmune serum from pigs that survive challenge with G. parasuis or rodent serum after exposure to G. parasuis. These methods are successful in identifying immunogenic proteins, but they are unable to distinguish the role those proteins play in protection.

As currently available methods of controlling Glasserella parasuis infections are limited in effectiveness, effective methods and compositions for prevention and treatment are needed. In particular, there is a need to identify proteins that are cross-protective and can permit the development of effective vaccines, in particular for prevention of infection by G. Parasuis are needed.

### SUMMARY

Provided herein are methods to identify immunogenic G. Parasuis polypeptides, preparation of said polypeptides as recombinant proteins, compositions comprising such recombinant proteins, kits comprising such recombinant proteins, and methods of using such recombinant proteins to prevent G. Parasuis in pigs.

The disclosure relates to a composition for prevention and/or amelioration of Glaesserella parasuis infection comprising at least one of recombinint G. parasuis Lipoprotein A (LppA), Thiamine biosynthesis protein ApbE, penicillin-binding protein activator LpoA, outer membrane protein assembly protein YaeT, catalase, D-alanyl-D-alanine carboxypeptidase fraction A (PBP5), or spermidine/putrescene ABC transporter ATPase protein (PotA), or a combination thereof, and a veterinarily-acceptable adjuvant or carrier.

In an embodiment, the disclosure relates to a composition comprising a recombinant G. parasuis Lipoprotein A with an amino acid sequence set forth in SEQ ID NO: 1, or a functional derivative thereof having 80% sequence identity to SEQ ID NO: 1 In an embodiment, the invention relates to a composition comprising a recombinant G. parasuis ApbE with an amino acid sequence set forth in SEQ ID NO: 2, or a functional derivative thereof having 80% sequence identity to SEQ ID NO:2. In an embodiment, the invention relates to a composition comprising a recombinant G. parasuis LpoA with an amino acid sequence set forth in SEQ ID NO: 3, or a functional derivative thereof having 80% sequence identity to SEQ ID NO: 3. In an embodiment, the invention relates to a composition comprising a recombinant G. parasuis YaeT with an amino acid sequence set forth in SEQ ID NO:4, or a functional derivative thereof having 80% sequence identity to SEQ ID NO: 4. In an embodiment, the disclosure relates to a composition comprising a recombinant G. parasuis catalase with an amino acid sequence set forth in SEQ ID NO: 7, or a functional derivative thereof having 80% sequence identity to SEQ ID NO: 7. In an embodiment, the disclosure relates to a composition comprising a recombinant G. parasuis PBP5 with an amino acid sequence set forth in SEQ ID NO: 5, or a functional derivative thereof having 80% sequence identity to SEQ ID NO: 5. In an embodiment, the disclosure relates to a composition comprising a recombinant G. parasuis PotA with an amino acid sequence set forth in SEQ ID NO: 6, or a functional derivative thereof having 80% sequence identity to SEQ ID NO: 6.

In an embodiment, the disclosure relates to a method for provoking an immune response in a pig, the method comprising administering to the pig at least one effective dose of a composition comprising at least one recombinant G. parasuis Lipoprotein A, Thiamine biosynthesis protein ApbE, penicillin-binding protein activator LpoA, outer membrane protein assembly protein YaeT, catalase, D-alanyl-D-alanine carboxypeptidase fraction A (PBP5), or spermidine/putrescene ABC transporter ATPase protein (PotA), or a combination thereof, and a veterinarily-acceptable adjuvant or carrier.

In an embodiment, the disclosure relates to a vector expressing a G. parasuis Lipoprotein A, Thiamine biosynthesis protein ApbE, penicillin-binding protein activator LpoA, outer membrane protein assembly protein YaeT, catalase, D-alanyl-D-alanine carboxypeptidase fraction A (PBP5), spermidine/putrescene ABC transporter ATPase protein (PotA), or a functional derivative thereof.

In an embodiment, the disclosure relates to a host cell expressing the vector expressing a G. parasuis Lipoprotein A, Thiamine biosynthesis protein ApbE, penicillin-binding protein activator LpoA, outer membrane protein assembly protein YaeT, catalase, D-alanyl-D-alanine carboxypeptidase fraction A (PBP5), spermidine/putrescene ABC transporter ATPase protein (PotA), or a functional derivative thereof.

### BRIEF DESCRIPTION OF THE DRAWINGS

FIG. 1A and FIG. 1B depict photographs of two-dimensional gel western blots of 12939 outer membrane proteins (OMPs) obtained with pooled day 42 serum. FIG. 1A shows OMPs detected in serum from Nagasaki bacterin-vaccinated animals. FIG. 1B shows OMPs detected in serum from HS069-vaccinated animals.

FIG. 2 depicts a photograph of outer membrane proteins separated by 2DGE and stained with SYPRO Ruby protein gel stain for visualization. Proteins identified with a 1; 2; 3; 4; 5; 6; 7; 9; 10; 11; 15; 16; 17; 18; 19; 20; 21; 22; 35; and 36 were identified on western blots probed with serum from HS069 bacterin-vaccinated (protected) animals, and proteins identified with a 8; 12; 13; 14; 23; 24; 25; 26; 27; 28; 29; 30; 31; 32; 33; 34; and 37 were identified on western blots probed with serum from Nagasaki bacterin-vaccinated (non-protected) animals.

FIG. 3A and FIG. 3B depict graphs of survival curves after homologous and heterologous challenge of animals vaccinated with adjuvant only, HS069 Bacterin, or Subunit vaccine Combination 3. FIG. 9A shows survival curves after G. parasuis HS069 (homologous) challenge. FIG. 9B shows survival curves after G. parasuis 12939 (heterologous) challenge. The X Axis shows days after vaccination; and the Y Axis shows percent survival. Survival of animals vaccinated with sham vaccine is indicated by filled circles (•); survival of animals vaccinated with bacterin vaccine is indicated by filled squares (▪); and survival of animals vaccinated with subunit vaccine Combination 3 is indicated by filled triangles (▴).

FIG. 4A to FIG. 4C depict graphs of the antibody titers to recombinant proteins within the Combination 3 subunit vaccine. FIG. 4A shows the reciprocal log₁₀ for rApbE. FIG. 4B shows the reciprocal log₁₀ for rLpoA. FIG. 4C shows the rYaeT. The Y Axis shows the calculated reciprocal log₁₀ for the different subunits. The X axis shows the days post vaccination (0; 21; and 43). Results for adjuvant only vaccinated-animals are indicated by filled circles (•); results for HS069 bacterin-vaccinated animals are indicated by filled squares (▪); and results for subunit-vaccinated animals are indicated by filled triangles (▴).

FIG. 5A and FIG. 5B depict graphs of G. parasuis antibody titers in animals vaccinated with adjuvant only, Bacterin vaccine, or Subunit vaccine (Combination 3). FIG. 5A shows results for G. parasuis HS069. FIG. 5B shows results for G. parasuis 12939. The Y Axis shows the calculated reciprocal log₁₀ for the different strains. The X axis shows the days post vaccination (0; 21; and 43). Results for sham-vaccinated animals are indicated by filled circles (•); results for bacterin-vaccinated animals are indicated by filled squares (▪); and results for subunit-vaccinated animals are indicated by filled triangles (▴).

FIG. 6 depicts ELISpot counts for each treatment group after stimulation with G. parasuis HS069 or a recombinant protein. Data for subunit vaccine (Combination 3) stimulated with ApbE is represented with filled up-pointing triangles (▴), data for bacterin vaccine stimulated with ApbE is represented by filled squares (▪), data for control vaccine stimulated with ApbE is represented with filled circles (•), data for subunit vaccine (Combination 3) stimulated with LpoA is represented with filled down-pointing triangles (▾), data for bacterin vaccine stimulated with LpoA is represented with empty squares (□) data for control vaccine stimulated with LpoA is represented with empty circles (∘), data for subunit vaccine (Combination 3) stimulated with YaeT is represented with empty up-pointing triangles (Δ), data for bacterin vaccine stimulated with YaeT is represented with half-filled squares ( ) data for control vaccine stimulated with YaeT is represented with half-filled circles ( ) data for subunit vaccine (Combination 3) stimulated with HS069 is represented with empty down-pointing triangles (∇), data for bacterin vaccine stimulated with HS0669 is represented with half-filled squares, and data for control vaccine stimulated with HS069 is represented with half-filled circles.

FIGS. 7A and 7B depict images of western blots assessing cross-reactivity between antigens in subunit vaccine (Combination 3) and HS069 bacterin vaccine. FIG. 7A shows an image of a blot probed with pooled day 43 subunit vaccine sera. FIG. 7B shows an image of a blot probed with pooled day 43 HS069 bacterin sera. The sample types are denoted at the top of the gel. M=protein size markers; 1=rLpoA, 2=rYaeT, 3=rApbE, and 4=G. parasuis HS069.

FIG. 8A and FIG. 8B depict graphs of the G. parasuis survival when day 43 serum from each vaccine group was added to activate the classical complement pathway. FIG. 8A shows the G. parasuis HS069 survival. FIG. 8B shows the G. parasuis 12939 survival. The Y axis shows the obtained Log₁₀ CFU/mL values. The X axis shows the different treatments: sham vaccine: adjuvant only; Bacterin vaccine: HS069; subunit vaccine: Combination 3 (LpoA, YaeT, AbpE). Significantly different values are represented with different letters (p<0.05).

FIG. 9 depicts a graph of the survival of G. parasuis 12939 when day 43 serum from each vaccine group was added to promote opsonization in whole blood. Pooled serum from day 0 is included as control. The Y axis shows the obtained Log₁₀ CFU/mL values. The X axis shows the different treatments: DO: prior to vaccination; Subunit: vaccine Combination 3 (LpoA, YaeT, AbpE); Bacterin: HS069; Sham: adjuvant only. Significantly different values are represented with different letters (p<0.05).

FIG. 10 depicts an image of an SDS-PAGE where lane 1 (L1) contains protein markers, lane 3 (L3) contains immunoprecipitated proteins isolated by preclearing with Nagasaki-vaccinated DO and probing with Nagasaki-vaccinated D42 (iteration 1); lane 6 (L6) contains immunoprecipitated proteins isolated by preclearing with HS069-vaccinated DO and probing with HS069-vaccinated D42 (iteration 2); lane 9 contains immunoprecipitated proteins isolated by preclearing with Nagasaki-vaccinated D42 and probing with HS069-vaccinated D42 (iteration 3), lanes not listed were run empty.

FIG. 11A to FIG. 11D depict graphs of the serum antibody titers for each of catalase, PBP5, and PotA, or challenge strain. FIG. 11A shows catalase antibody titers. FIG. 11B shows PBP5 antibody titers; FIG. 11C shows PotA antibody titers, and FIG. 11D shows antibody titers of challenge strain. Titers from subunit (Combination 4)-vaccinated animals are represented by circles (•) and titers from adjuvant-only vaccinated animals are represented by triangles (▴). The Y Axis shows the Log₁₀ of the antibody titer. The X Axis shows the days post treatment.

FIG. 12 depicts a graph of the survival post challenge of animals vaccinated with a mixture of catalase, PBP5, and PotA (Combination 4) (5/7) and non-vaccinated animals (3/8).

FIG. 13A to FIG. 13D depict graphs of antibody titers after homologous challenge of animals vaccinated with Combination 1 or adjuvant alone. FIG. 13A shows LpoA titers. FIG. 13B shows YaeT titers. FIG. 13C shows Lipoprotein A titers. FIG. 13D shows HS069 titers. The X Axis shows days after vaccination, and the Y Axis shows the Log₁₀ reciprocal IgG titer. Titers in animals vaccinated with the Combination 1 subunit vaccine are indicated by filled triangles (▴); and titers in animals vaccinated with adjuvant only are indicated by filled circles (•).

FIG. 14 depicts a graph of survival curve after homologous challenge of animals vaccinated with Combination 1 or adjuvant alone. The X Axis shows days after vaccination; and the Y Axis shows percent survival. Survival of animals vaccinated with adjuvant only is indicated by filled squares (▪); and survival of animals vaccinated with Combination 1 subunit vaccine is indicated by filled triangles (▴).

FIG. 15A to FIG. 15D depict graphs of antibody titers after heterologous challenge of animals vaccinated with Combination 1 or adjuvant alone. FIG. 15A shows LpoA titers. FIG. 15B shows YaeT titers. FIG. 15C shows Lipoprotein A titers. FIG. 15D shows 12939 titers. The X Axis shows days after vaccination, and the Y Axis shows the Log₁₀ reciprocal IgG titer. Titers in animals vaccinated with the Combination 1 subunit vaccine are indicated by filled circles (•); and titers in animals vaccinated with adjuvant only are indicated by filled squares (▪).

FIG. 16 depicts a graph of survival curves after heterologous challenge of animals vaccinated with Combination 1 or adjuvant alone. The X Axis shows days after vaccination; and the Y Axis shows percent survival.

FIG. 17A to FIG. 17D depicts graphs of antibody titers after homologous challenge of animals vaccinated with Combination 2 or adjuvant alone. FIG. 17A shows catalase titers.

FIG. 17B shows PBP5 titers. FIG. 17C shows Lipoprotein A titers. FIG. 17D shows HS069 titers. The X Axis shows days after vaccination, and the Y Axis shows the Log₁₀ reciprocal IgG titer. Titers in animals vaccinated with the Combination 2 subunit vaccine are indicated by filled circles (•); and titers in animals vaccinated with adjuvant only are indicated by filled triangles (▴).

FIG. 18 depicts a graph of survival curves after heterologous challenge after vaccination with Combination 2 or adjuvant alone. The X Axis shows days after vaccination; and the Y Axis shows percent survival.

FIG. 19 depicts a graph of the survival post-challenge of animals vaccinated with LppA, Bacterin, LppA+Bacterin, or adjuvant alone.

### BRIEF DESCRIPTION OF THE SEQUENCE LISTING

The amino acid sequences disclosed in the specification are listed in Table 1, below.

TABLE 1

| SEQ ID | Protein | Sequence |
|---|---|---|
| SEQ ID | LppA | GGGSSGGHQQAVDKTENPITNQQVSENNPQ |
| NO: 1 | | SLDKNNASKAPEAKEDKQIKEPIVGKQSKE |
| | | KIEPPSQKLNDQDIWQGFSKNGGYYDASNW |
| | | VSITLEGKEIKLAPVNGSNSNNSKMNTLRD |
| | | ADGNLVGYYGYATLSKTKPDPYRPDENNVE |
| | | RKYMALQEVTAREEAKTRPTHDLSYQGKMY |
| | | YALKDAPEQAKEANVTATYKHMNKSISINI |
| | | YGVNGNDLTTNQTNVSENGYIFTRLYLMGK |
| | | PKIPAGQFDGGLYGKSGEILLGKAKNEDAT |
| | | PHTPQKNWEGVIGATATKDK |
| SEQ ID | ApbE | CNKEPQQIAFEGKTMGTTYHIKYIDDGSLT |
| NO: 2 | | NLPKPEEVQRQMDTLLKMVNNEMSTYQKDS |
| | | QISQFNQMKEADKPFEIASDFAKVVKEAVR |
| | | LNKVTEGALDVTVGPLVNLWGFGPDKRLNK |
| | | APTEEQIAERATWVGIEKLVLDDNATKATL |
| | | TKKVPELYLDLSAIAKGFGVDKLADYLEQL |
| | | GVQHYLVEIGGELRGKGLNLKGQPWRIAVE |
| | | KPEFAQGTSSQIVVPLHNLGMATSGNYRNY |
| | | FEDEQGNRLSHIIDPKALRPISHNLASITV |
| | | FAPTTMTADGLSTGLYVLGAEKALEVAERE |
| | | NLAIFLIIKKGDGYETKMSSAFEKLINQK |
| SEQ ID | LpoA | CSGNKVTETLKNEAYGNSEFYINKAEQSRN |
| NO: 3 | | IEEQQSYRLLAVRKLIEENKVVEAQNTFSE |
| | | ILVAKLNDEQKLEHRLLIAQLAALQGNNSE |
| | | AQGVLRALPQTLLSQSQRLRVYQTQARIAE |
| | | NQNDVIAAVNARALMDRYMTDTQSRQANND |
| | | KIWEMLRNANRGMLEKAVAGPGEIGLAGWL |
| | | ALVVAYNQNMSNPAQLPQVIEMWKQQYPNH |
| | | SAVLLLPIELRNVSSFQQTQLNGVALLLPL |
| | | SGDAKILGEIIKRGFDDAKGTTTMQVQVFD |
| | | TDSAPINDLLMQAKQQGAQTIIGPLLKPRV |
| | | DEMLTSPEISNINVLALNSTPNVRAVAKVC |
| | | YYGLSPEAEARSAAERFLKDGLQHAVVVAP |
| | | NGDFGTRSAEAFAQRWRQLTNRDTDIRYYN |
| | | QAFEVTSLLQGSGIGQGSGLYALGTAEQLS |
| | | DLKQSLDNSPLANQFPIYTSSRSNSPNNGP |
| | | DFRLTMEGVKFSEIPLLSDPSSSEYKKAEQ |
| | | IVESDFSMMRLYAMGSDTWSIANKFNEFRQ |
| | | IPGYKVHGLTGVLSAGPNCNIEREMNWLQY |
| | | RGGSIVDAN |
| SEQ ID | YaeT | SPFVIKDIRIDGIQTSVSQTMIPTLPVKVG |
| NO: 4 | | QLATDNDIANVIRHLFVQNRFQNISATREG |
| | | DILIIKVVENPVINNVDIEGNSAIPKEPLE |
| | | QNLKANLITKGELFNSTKLDAFKSALIEHY |
| | | HSIGRYNATIDTVITNSDNGGVNIKLDIKE |
| | | GDVAYVKAINFEGNQAFTSKVLMKQLDIQP |
| | | DVSWFNIFQSSKFEQTAYNQDLENLRNFYM |
| | | NQGYAKFNVEAEQPVFSDDKKEVSLTYKIN |
| | | EGIPYTVSEIRIVGNTAKLDNELNEQLKYY |
| | | KTGELFSKNELSKIEDNIKQVFGNNGFASA |
| | | KIELHPTFDDTNKTIRLTFVVDAGQRVYVR |
| | | QIRFEGNDVTADSTLRREMRQQEGAWLSTS |
| | | LAALGKARLERTGFYDSVEMSYPNVPNTAD |
| | | QVDIVYKIKERNTGSINFGVGYGTESGLSY |
| | | QAGIKQDNFLGMGSTISLNGTRNDYGTSIN |
| | | LGYTEPYFTKDGVSLGGNIFYEDYDNSKNN |
| | | SVASYKRQTYGVNSTLGFPVNEYNSYFLGL |
| | | GYTQDLIKNARREFNRELYVQSMKFPIDND |
| | | LNVYKKIKANDFDFSFGWNYNSLNRGFLPT |
| | | EGTLTSISGRITTPGSDNKYYKVSIDFKNY |
| | | LPVNREHKWVISTKANIAYANGFSGKELPF |
| | | YQTYTAGGIGSLRGFAYGAIGPKAIYLGKD |
| | | NIRFDSVGDIVGGNAMASATLELITPTPFV |
| | | SEKYQHNVRTSLFVDAATVLNTKWKTNPVV |
| | | NDIQYNYSSLENFKDYKRFRASTGIAFQWN |
| | | SPIGPLVFSYAKPIKKYQGDEIEQFQFSIG |
| | | GSF |
| SEQ ID | PBP5 | QDIDYGIAAPQLNAQTYILMDYNSGAVLAS |
| NO: 5 | | LNPDQRQAPASLTKMMTSYVVGEALKTGKI |
| | | KNSDLVTVTENSWGQKFPGSSKMFLNVNQQ |
| | | VSVEDLNKGIIVVSGNDACVAIAEHISGSE |
| | | QTFIDSMNKFVQKFGLKNTHFATVHGLDHP |
| | | EQYSSARDMAIIGAHIIRDLPEEYKIYSEK |
| | | EFTYNKITQPNRNGLLWDKSINVDGMKTGH |
| | | TDQAGYNLVASAYNSNTRLISVVMGVPTYK |
| | | GREVESKKLLQWGFANFETVKPLSAGQSVL |
| | | EQAVYYGDLSKAQLGTLQDSYITVPKGKTA |
| | | ELKARYTLEKTNLEAPLVKGQVVGKVIYQL |
| | | NGKDVASVDLQVMHDVQEAGIFGKAWDWIV |
| | | LTVKGLFN |
| SEQ ID | PotA | EKLEQKPIIELRSITKRYGEKTIIDGLDLT |
| NO: 6 | | INDGEFLTILGPSGCGKTTVLRLIAGFEDL |
| | | TDGSIILDGQDVSNVDAEQRPVNTVFQSYA |
| | | LFPHMTIFDNVAFGLRMQKVPEAEIKPRVM |
| | | EALRMVRLEEMAPRKPSQLSGGQQQRIAIA |
| | | RAVVNQPKVLLLDESLSALDYKLRKEMQHE |
| | | LKQLQRQLGITFIFVTHDQEEALTMSDRII |
| | | VMNKGKIQQDGSPREIYEEPSNLFVARFIG |
| | | EINVFDATVIERVTEKTVKANVEGRICEIH |
| | | TELPVHPNQKLKVLLRPEDIVIEELDENES |
| | | AKAVIGHVSDRTYKGMTLESNVVLDHNQMK |
| | | VLVSEFFNEDDPHMDHSVGQKVALTWHEGW |
| | | EVVLDDEDC |
| SEQ ID | Catalase | YKCPFDHGSKTLTTAAGAPVVDNDNTMSAG |
| NO: 7 | | PRGPLLLQDVWFQEKLAHFARERIPERVVH |
| | | AKGSAAYGTFTVTADITKYTKAAVFKPGAQ |
| | | TEVLLRFSTVAGERGAADAERDVRGFSLKF |
| | | YTEQGNWDLVGNNTPVFFIRDPLKFPDFIH |
| | | TQKRNPQTNLRDANAAWDFWSRHPESMHQI |
| | | MTLFSDRGIPATLRHMNGYGSHTYSFVNAN |
| | | NERFWVKFHFKTQQGHKFFTNEEAAKVVGE |
| | | DRESSQKDLYEAIERGEFPRWTVQVQIMPE |
| | | ADAHKHNYAFDLTKVWPHADYPVIEVGVLE |
| | | LNRNPQNYFAEVEQAAFAPSNIVPGIGFSP |
| | | DRMLQGRLFSYQDAQRYRLGVNHHQIPVNA |
| | | PKCSYHTTHRDGAMRVDANGGNHPNYAPNR |
| | | FDTYVPTHDQLPLQIEREAAHFNFREYDED |
| | | YYSQPAALYNLFTAEEKDRLAGNFAAGLSG |
| | | VTIPEIVERQMAHFEKVSPELANAIRQKLA |
| | | K |

### DETAILED DESCRIPTION

The present disclosure relates to methods for identifying immunogenic G. Parasuis polypeptides, compositions comprising such polypeptides, kits comprising such polypeptides, and methods of using such polypeptides and compositions to prevent G. Parasuis in pigs.

Glaesserella parasuis is a Gram negative pathogen that causes arthritis, polyserositis and meningitis in pigs. It is globally distributed and a significant cause of losses to the swine industry. G. parasuis isolates are classified by their capsular polysaccharide into 15 serovars.

Either commercial or autogenous vaccines can be used to control G parasuis infection, although their efficacy has been variable. The broad range of potentially pathogenic serovars and genotypes has impaired the development of a universal vaccine for G parasuis. Homologous protection between isolates from the same serovar group is relatively satisfactory, whereas heterologous protection is restricted to a few serovars. Several “universal” (independent of serovar) vaccine prototypes have been experimentally developed but are not commercially available.

Developing effective vaccines against G. parasuis has been complicated by the generation of strain or serovar specific immunity. To combat this, resources have been directed at identifying and testing subunit vaccine candidates. In this disclosure, novel comparative methods were utilized to identify proteins of interest involved in the protective immune response to heterologous challenge with G. parasuis 12939. Using 2D gel electrophoresis (2DGE) and immunoblotting, three proteins were identified as components of the protective immune response but lacking in the non-protective immune response (ApbE, LpoA, and YaeT). These were selected for in vivo testing; however, a combination of the three did not afford improved survival over sham-vaccinated controls when challenged with G. parasuis HS069 or 12939. Immunogenic proteins were also identified through immunoprecipitation using serum with known potential to provide heterologous protection. From the identified proteins, three (catalase, PBP5, and PotA) were selected and evaluated as vaccine antigens to protect pigs against G. parasuis disease. Also tested were subunit vaccines comprising LppA, LpoA, and YaeT (Combination 1) and catalase, PBP5, and LppA (Combination 2).

To better understand the protective immune response, the cell-mediated and antibody responses were assessed using several immunologic assays. The cytokine response by PBMCs stimulated with G. parasuis HS069 whole cell sonicate was similar between subunit and bacterin vaccinated animals. Antibody responses were elevated to the recombinant proteins in subunit-vaccinated animals and to the G. parasuis challenge strains in the bacterin-vaccinated animals. While there was an elevation in antibody titer of subunit-vaccinated animals to G. parasuis 12939 over that of the sham-vaccinated controls, the titers of bacterin-vaccinated animals were significantly higher and provided better protection against challenge.

The differences in protection between subunit-vaccinated and bacterin-vaccinated animals seen here may be attributed to differences in antibody titers to G. parasuis whole cell sonicate. It appears that a minimum threshold must be met to confer protection against challenge, as lower levels of pathogen-specific antibody are unable to sufficiently induce complement fixation or opsonization in whole blood. However, while quantity of G. parasuis specific antibody appears to define the differences in protection for subunit and bacterin vaccinated animals, it does not explain the differences in protection between Nagasaki bacterin-vaccinated animals and HS069 bacterin-vaccinated animals previously described (Hau S J, et al., 2021,” Importance of strain selection in the generation of heterologous immunity to Glaesserella (Haemophilus) parasuis) Vet. Immunol. Immunopathol. 234:110205). These animals had similar titers to G. parasuis 12939 whole cell sonicate, though Nagasaki bacterin-vaccinated animals were not protected against 12939 challenge.

Forty six (46) unique antigens associated with heterologous protection against G. parasuis were identified employing the immunoprecipitation described herein. Of the 46 antigens, six had previously been identified as potential vaccine antigens (catalase, dihydrolipoamide dehydrogenase, heme binding protein A, PBP5, PotA, transferrin binding protein). Three of these, which have not previously been used with G. parasuis, were selected for use in a vaccine trial: catalase, PBP5, PotA (Combination 4). Catalase breaks down hydrogen peroxide thereby protecting the bacterial cell from damage (L. Gebicka and J. Krych-Madej, 2019, “The role of catalases in the prevention/promotion of oxidative stress,” J. Inorg. Biochem. 197:110699). It has shown efficacy as a vaccine antigen in other Gram negative bacterial species including Helicobacter pylori and Pseudomonas aeruginosa (L. D. Thomas, et al., 2000, “Catalase immunization from Pseudomonas aeruginosa enhances bacterial clearance in the rat lung,” Vaccine 19 (2-3): 348-357; and F. J. Radcliff, et al., 1997, “Catalase, a novel antigen for Helicobacter pylori vaccination,” Infect. Immun. 65 (11): 4668-4674). PBP5 (D-alanyl-D-alanine carboxypeptidase fraction A) is a carboxypeptidase that functions in peptidoglycan formation and is important for normal cellular morphology (E. Sauvage, et al., 2008, “The penicillin-binding proteins: structure and role in peptidoglycan biosynthesis,” FEMS Microbiol. Rev. 32 (2): 234-258). PBP5 of Enterococcus has shown efficacy as an antigen in opsonophagocytic evaluation and in assessment of bacteremia reduction in mice (F. Romero-Saavedra, et al., 2014, “Identification of peptidoglycan-associated proteins as vaccine candidates for enterococcal infections,” PLOS One 9 (11): e111880). Finally, PotA (spermidine/putrescene ABC transporter ATPase protein) is part of a highly conserved protein transporter that imports polyamines into the cell (M. L. Di Martino, et al., 2013, “Polyamines: emerging players in bacteria-host interactions,” Int. J. Med. Microbiol. 303 (8): 484-491). Polyamine uptake has been shown to play a role in virulence in several bacterial species, including G. parasuis (M. L. Di Martino, Supra; and K. Dai, Z., et al., 2021, “Deletion of Polyamine Transport Protein PotD Exacerbates Virulence in Glaesserella (Haemophilus) parasuis in the Form of Non-biofilm-generated Bacteria in a Murine Acute Infection Model,” Virulence 12 (1): 520-546). While PotA had not been previously used as a vaccine antigen, PotD, the periplasmic binding protein for the ABC transporter, has shown efficacy in mouse models of disease for G. parasuis and Streptococcus pneumoniae (P. Shah and E. Swiatlo, 2006, “Immunization with polyamine transport protein PotD protects mice against systemic infection with Streptococcus pneumoniae,” Infect. Immun. 74 (10): 5888-5892).

Tested as subunit vaccines were also combination of recombinant LppA, LpoA, and YaeT (Combination 1), and catalase, PBP5, and LppA (Combination 2). Further, recombinant LppA was tested alone or in combination with bacterin. LppA alone provided slightly better protection than bacterin alone, and the LppA/bacterin combination provided even better protection.

Preparation of vaccines which contain peptide sequences as active ingredients are well understood in the art. Typically, such vaccines are prepared as injectables, either as liquid solutions or suspensions; solid forms suitable for solution or suspension in liquid prior to injection may also be prepared. The preparation may also be emulsified. The active immunogenic ingredient is often mixed with excipients which are pharmaceutically acceptable and compatible with the active ingredient. Suitable excipients are, for example, water, saline, dextrose, glycerol, ethanol, or the like and combinations thereof. In addition, if desired, the vaccine may contain minor amounts of auxiliary substances such as wetting or emulsifying agents, pH buffering agents, or adjuvants which enhance the effectiveness of the vaccine. The vaccines are conventionally administered parenterally, by injection, for example, either subcutaneously or intramuscularly. Additional formulations which are suitable for other modes of administration include suppositories and, in some cases, intranasal application or oral formulations. For suppositories, traditional binders and carriers may include, for example, polyalkylene glycols or triglycerides; such suppositories may be formed from mixtures containing the active ingredient in the range of 0.5% to 10%, preferably 1-2%. Formulations for intranasal administration include nasal drops, aerosols (liquid or dry powder), While the choice of vaccine formulations, mucoadhesives, mucosal and epithelial permeation enhancers, and ligands that target M-cells are important, safe and effective intranasal mucosal vaccine adjuvants are needed to successfully develop an intranasal vaccine that is not based on live-attenuated viruses or bacteria. Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain 10%-95% of active ingredient, preferably 25-70%.

The amino acid sequences identified herein include their pharmaceutically acceptable salts, including the acid addition salts (formed with the free amino groups of the peptide) and which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The vaccines taught herein can be administered in a manner compatible with the dosage formulation, and in such amount as will be prophylactically or therapeutically effective and immunogenic. The quantity to be administered can depend on the subject to be treated, capacity of the subject's immune system to synthesize antibodies, and the degree of protection desired. Precise amounts of active ingredient required to be administered can depend on the judgment of the care-giver/practitioner. Suitable dosage ranges for subcutaneous or intramuscular injection can be, for example, about 1 μg to about 10 mg active ingredient per subject. For other types of administration, dosages can range, illustratively, from about 10 μg to about 100 mg. Suitable regimes for initial administration and booster shots are also variable, but are typified by an initial administration followed in about two to about three week intervals by a subsequent injection or other administration.

For topical administration, the composition can be formulated in the form of an ointment, cream, gel, lotion, drops (such as eye drops and ear drops), or solution (such as mouthwash). Wound or surgical dressings, sutures and aerosols may be impregnated with the composition. The composition may contain conventional additives, such as preservatives, solvents to promote penetration, and emollients. Topical formulations may also contain conventional carriers such as cream or ointment bases, ethanol, or oleyl alcohol.

The subunit vaccines described herein can also be administered with a suitable adjuvant in an amount effective to enhance the immunogenic response. For example, suitable adjuvants may include alum (aluminum phosphate or aluminum hydroxide), which is used widely in humans, and other adjuvants such as saponin and its purified component Quil A, Freund's complete adjuvant, RIBBI adjuvant, and other adjuvants used in research and veterinary applications. Examples of other chemically defined preparations include muramyl dipeptide, monophosphoryl lipid A, phospholipid conjugates, encapsulation of the conjugate within a proteoliposome, and encapsulation of the protein in lipid vesicles.

Accordingly, the subunit vaccines described herein will provide methods for preventing G. Parasuis infection in a pig when an effective amount of the subunit vaccine composition is administered to the pig, wherein the effective amount is sufficient to prevent infection by the bacteria. As would be recognized by one of ordinary skill in this art, the amount of subunit vaccine needed to be effective in preventing infection will vary depending on the nature and condition of the subject, and/or the severity of any preexisting infection.

In another aspect, the present description provides a kit for preventing G. parasuis infection. In one embodiment, the kit comprises the isolated cross-reactive proteins described herein or fragments thereof in a suitable form, such as lyophilized in a single vessel.

Unless otherwise explained, all technical and scientific terms used herein have the same meaning as commonly understood by one of ordinary skill in the art to which this disclosure belongs.

The singular terms “a”, “an”, and “the” include plural referents unless context clearly indicates otherwise. Similarly, the word “or” is intended to include “and” unless the context clearly indicate otherwise.

As used herein, the term “about” is defined as plus or minus ten percent of a recited value. For example, about 1.0 g means 0.9 g to 1.1 g.

As used herein, the term “adjuvant” means a pharmacological or immunological agent that modifies the effect of other agents, such as a drug or immunogenic composition. Adjuvants are often included in immunogenic compositions to enhance the recipient's immune response to a supplied antigen. See below for a further description of adjuvants.

As used herein, the terms “prevent”, “preventing”, “prevention”, and the like, are used to mean to inhibit the replication of a microorganism, to inhibit transmission of a microorganism, or to inhibit a microorganism from establishing itself in its host. These terms, and the like, can also mean to inhibit or block one or more signs or symptoms of infection.

As used herein, the terms “therapeutically effective amount” and “effective amount” mean an amount of an active ingredient, e.g., a composition according to the disclosure, with or without an adjuvant, as appropriate under the circumstances, provided in a single or multiple doses as appropriate, sufficient to effect beneficial or desired results when administered to a pig. An effective amount can be administered in one or more administrations, applications or dosages. A therapeutically effective amount of a composition according to the invention may be readily determined by one of ordinary skill in the art, and provides a measurable benefit to a patient, such as protecting the animal from subsequent challenge with a similar pathogen.

As used herein, the term “veterinarily-acceptable carrier”, refers to substances which are, within the scope of sound medical judgment, suitable for use in contact with the tissues of animals, without undue toxicity, irritation, allergic response, and the like, commensurate with a reasonable benefit-to-risk ratio, and effective for their intended use.

As used herein, the term “homologous” refers to the level of similarity between two or more polynucleotide and/or polypeptide sequences in terms of percent of positional identity, i.e. sequence similarity or identity. Homology also refers to the concept of similar functional properties among different polynucleotides or polypeptides. Thus, the compositions and methods disclosed further comprise homologues to the polynucleotide sequences and polypeptide sequences taught herein. “Orthologous,” as used herein, refers to homologous nucleotide sequences and/or amino acid sequences in different species that arose from a common ancestral gene during speciation. A homologue of a polynucleotide sequence or a polypeptide disclosed herein has a substantial sequence identity, e.g. at least about 80%, 81%, 82%, 83%, 84%, 85%, 86%, 87%, 88%, 89%, 90%, 91%, 92%, 93%, 94%, 95%, 96%, 97%, 98%, 99%, and/or 100%, to said polynucleotide or polypeptide sequence.

Mention of trade names or commercial products in this disclosure is solely for the purpose of providing specific information and does not imply recommendation or endorsement by the U.S. Department of Agriculture. For the purposes of this disclosure, the following are defined:

As used herein, “Bind-silane” refers to 3-trimethoxysilylpropyl methacrylate, and is used to covalently attach polyacrylamide gels to a glass surface. The gel stays firmly attached to the glass during staining and drying procedures.

As used herein, “10X Bolt™ Sample Reducing Agent” is a chemical used to reduce protein samples for protein electrophoresis. At a 10× concentration, it contains 500 mM dithiothreitol (DTT) in a stabilized liquid form. “Bolt” is a registered trademark currently held by Thermo Fisher Scientific.

ChemiDoc™ MP Imaging System is an instrument for imaging and analyzing gels and western blots. ChemiDoc™ is a trademark of ANALYTIK JENA US LLC.

As used herein, “DTT” refers to dithiothreitol, a small-molecule redox reagent also known as Cleland's reagent.

As used herein, the Proxeon Easy-nLC system is an automated liquid chromatograph for proteomic analyses featuring integrated hardware, autosampler, and a computer.

As used herein, “EDTA” refers to ethylenediaminetetraacetic acid, an aminopolycarboxylic acid that chelates ions forming water-soluble complexes.

As used herein, “Emulsigen-D” refers to an oil in water adjuvant emulsion free of animal origin ingredients. Emulsigen is a registered trademark of Phibro Animal Health Corp.; Teaneck, New Jersey, USA.

As used herein, an “Ettan™ DALTsix Electrophoresis units” are systems designed for separation of discrete quantifiable protein spots. Ettan™ is a registered trademark for chemicals for use in chromatography and electrophoresis currently owned by Cytva Sweeden A B; Upsala, Sweeden.

As used herein, Geneious™ is a software for performing gene sequencing research, visualization, and analysis. Geneious™ is a registered trademark of Biomatters Ltd.; Auckland, New Zealand.

As used herein, GraphPad Prism™ is a computer software for analyzing and graphic scientific data. GraphPad Prism™ is a registered trademark of GraphPad Software LLC; La Jolla, California, USA.

As used herein, “iBlot™” refers to chemical reagents for non-medical purposes, for life sciences research. The iBlot™ Gel Transfer Stacks are disposable stacks that have integrated PVDF or nitrocellulose transfer membranes to perform dry blotting of protein. The iBlot™ Gel Transfer Stacks are used to transfer proteins using the iBlot™ Gel Transfer Device, Each iBlot™ Transfer Stack contains a copper electrode and appropriate cathode and anode buffers in the gel matrix to allow fast, reliable transfer of proteins or DNA. The iBlot™ Transfer Device is a dry transfer device that performs western blotting transfer efficiently and reliably without the need for liquid buffers. The iBlot™ Transfer Device is an integral part of the iBlot™ dry blotting system, which consists of the transfer device and consumable transfer stacks that contain the required buffers and transfer membrane (nitrocellulose or PVDF). “iBlot” is a registered trademark currently held by Thermo Fisher Scientific.

As used herein, Immulon™ 2 are small volume, flat bottom plates designed for use with colorimetric assays. Immulon™ is a registered trademark currently owned by Thermo Fisher Scientific; Roskilde, Denmark.

As used herein, “Immunospot™” refers to medical diagnostic reagents and plates. Immunospot™ is a registered trademark of Cellular Technology LLC, Shaker Heights, Ohio, USA.

As used herein, “IPG Buffers” refer to ampholyte-containing buffer concentrates specifically formulated for use with Immobiline™ DryStrip gels. Immobiline™ is a registered trademark currently owned by Cytva Sweeden AB; Upsala, Sweeden.

As used herein, “Immobiline™ DryStrip gels” and “IPG strips” refer to gels used for isoelectric focusing (IEF).

As used herein, “LTQ OrbiTrap™ Velos Pro” is a hybrid mass spectrometer, using tandem mass spectrometry in conjunction with the high resolution mass measurements of the molecular weight of the molecules. OrbiTrap™ is a registered trademark of Thermo Finnigan, LLC; San Jose, California, USA.

MS Amanda is a tool to identify peptide sequences in tandem mass spectra (MS/MS). The MS Amanda algorithm can use HCD, ETD, CID data, and can work as a plugin for the Proteome Discoverer platform.

As used herein, “NuPAGE™ LDS Sample Buffer” is a protein preparation buffer which comprises lithium dodecyl sulfate pH 8.4, Coomassie G250 and Phenol Red as tracking dyes, in glycerol. “NuPAGE” is a registered trademark currently held by Thermo Fisher Scientific.

PicoTip™ refers to a mass spectrometry sample-delivery device. PicoTip™ is a registered trademark of New Objective, Inc; Littleton, Massachusetts, USA.

Pierce™ is a house trademark for chemicals used in research registered by Pierce Biotechnology Inc.; Santa Clara, California, USA.

As used herein, “Sequest™ is a computer software for performing biomolecular analysis by analyzing data from spectrometers. Sequest™ is a registered trademark of the University of Washington, Seattle, Washington, USA.

As used herein, “StartingBlock™ (TBS) Blocking buffer” refers to a buffer containing a single purified protein for fast blocking of western blots and ELISA assays. “StartingBlock™” is a trademark currently held by Thermo Fisher Scientific.

As used herein, “SuperSignal™” refers to reagents and kits for use in chemiluminescence detection of substances on immunoblots. “SuperSignal” is a registered trademark of Pierce Biotechnology, Inc., Rockford, Illinois, USA.

SYPRO™ Ruby protein gel stain is a ready-to-use, ultrasensitive, luminescent stain for detection of proteins separated by polyacrylamide gel electrophoresis. SYPRO is a registered trademark of Molecular Probes, Inc.; Eugene, Oregon, USA.

As used herein, “TBS” and “Tris buffered saline” are used interchangeably and refer to an isotonic and non-toxic buffer capable of maintaining the pH within a relatively narrow range. TBS is composed of tris and sodium chloride, with the pH adjusted depending on the intended application.

TE 77 PWR system is a semi-dry transfer unit available from Cytva.

As used herein, “TE buffer” refers to a buffer solution containing Tris and EDTA.

As used herein “Tris” refers to tris(hydroxymethyl)aminomethane.

As used herein, “TRITON™ X-114” relates to a nonionic surfactant that has the chemical formula 2-[4-(2,4,4-trimethylpentan-2-yl) phenoxy]ethanol. “TRITON™ X-114” is compatible with anionic, cationic, and other nonionic detergents, and is chemically stable in most acidic and alkaline solutions. “TRITON” is a registered trademark for nonionic surfactants currently held by Thermo Fisher Scientific (Waltham, Massachusetts, USA).

As used herein, “TWEEN™ 20” refers to a non-ionic surfactant also known as polysorbate 20 or polyethylene glycol sorbitan monolaurate, which is also found under other commercial names. Polysorbate 20 is formed by the ethoxylation of sorbitan monolaurate. Its stability and relative nontoxicity allows it to be used as a detergent and emulsifier in a number of domestic, scientific, and pharmacological applications. As the name implies, the ethoxylation process leaves the molecule with 20 repeat units of polyethylene glycol. “TWEEN™ 20” is a trademark currently owned CRODA Americas LLC; Winterthur, Switzerland.

As used herein, UniProt™ proteome is a database containing sequences of protein sets for species with sequenced genomes from across the tree of life. UniProt™ is a registered trademark of the European Molecular Biology Laboratory Incorporated Association; Heidelberg, Federal Republic of Germany.

As used herein, BD Vacutainer™ are serum separator tubes are tubes for the collection of blood and serum separation. BD Vacutainer™ is a registered trademark of Becton Dickinson; Franklin Lakes, New Jersey, USA.

As used herein, “WedgeWell™” refers to reagent kits consisting primarily of electrophoresis gels for scientific research. WedgeWells™ have a wedge-shaped well which provides a higher loading capacity than other gels. WedgeWell™ 10% Tris-Glycine Gels are precast gels used for the protein separation of a wide variety of sample types. WedgeWell is a registered trademark currently held by Thermo Fisher Scientific.

The term “treating,” as used herein, refers to ameliorating, improving or remedying a disease, disorder, condition or symptom of a disease, disorder, or condition.

The term “preventing” means to stop or hinder a disease, disorder, condition, or symptom of a disease, disorder, or condition.

Embodiments of the present invention are shown and described herein. It will be obvious to those skilled in the art that such embodiments are provided by way of example only. Numerous variations, changes, and substitutions will occur to those skilled in the art without departing from the invention. Various alternatives to the embodiments of the invention described herein may be employed in practicing the invention. It is intended that the included claims define the scope of the invention and that methods and structures within the scope of these claims and their equivalents are covered thereby. All publications, patents, and patent applications mentioned in this disclosure are herein incorporated by reference to the same extent as if each individual publication, patent, or patent application was specifically and individually indicated to be incorporated by reference. In addition the disclosure encompasses any possible combination that also specifically excludes any one or some of the various embodiments and characteristics described herein and/or incorporated herein.

The amounts, percentages and ranges disclosed herein are not meant to be limiting, and increments between the recited amounts, percentages and ranges are specifically envisioned as part of the invention. All ranges and parameters disclosed herein are understood to encompass any and all subranges subsumed therein, and every number between the endpoints. For example, a stated range of “1 to 10” should be considered to include any and all subranges between (and inclusive of) the minimum value of 1 and the maximum value of 10 including all integer values and decimal values; that is, all subranges beginning with a minimum value of 1 or more, (e.g., 1 to 6.1), and ending with a maximum value of 10 or less, (e.g. 2.3 to 9.4, 3 to 8, 4 to 7), and finally to each number 1, 2, 3, 4, 5, 6, 7, 8, 9, and 10 contained within the range.

The term “consisting essentially of” excludes additional method (or process) steps or composition components that substantially interfere with the intended activity of the method (or process) or composition, and can be readily determined by those skilled in the art (for example, from a consideration of this disclosure or practice of the material disclosed herein).

According to MPEP 2173.05 (i), the current view of the courts is that there is nothing inherently ambiguous or uncertain about a negative limitation. So long as the boundaries of the patent protection sought are set forth definitely, albeit negatively, the claim complies with the requirements of 35 U.S.C. 112 (b) or pre-AIA 35 U.S.C. 112, second paragraph.

Any negative limitation or exclusionary proviso must have basis in the original disclosure. If alternative elements are positively recited in the specification, they may be explicitly excluded in the claims. See In re Johnson, 558 F.2d 1008, 1019, 194 USPQ 187, 196 (CCPA 1977) (“[the] specification, having described the whole, necessarily described the part remaining.”). See also Ex parte Grasselli, 231 USPQ 393 (Bd. App. 1983), aff′d mem., 738 F.2d 453 (Fed. Cir. 1984). In describing alternative features, the applicant need not articulate advantages or disadvantages of each feature in order to later exclude the alternative features. See Inphi Corporation v. Netlist, Inc., 805 F.3d 1350, 1356-57, 116 USPQ2d 2006, 2010-11 (Fed. Cir. 2015). The mere absence of a positive recitation is not basis for an exclusion. However, a lack of literal basis in the specification for a negative limitation may not be sufficient to establish a prima facie case for lack of descriptive support. Ex parte Parks, 30 USPQ2d 1234, 1236 (Bd. Pat. App. & Inter. 1993). “Rather, as with positive limitations, the disclosure must only ‘reasonably convey [ ] to those skilled in the art that the inventor had possession of the claimed subject matter as of the filing date.’ . . . . While silence will not generally suffice to support a negative claim limitation, there may be circumstances in which it can be established that a skilled artisan would understand a negative limitation to necessarily be present in a disclosure.” Novartis Pharms. Corp. v. Accord Healthcare, Inc., 38 F.4th 1013, 2022 USPQ2d 569 (Fed. Cir. 2022) (quoting Ariad Pharm. Inc. v. Eli Lilly & Co., 589 F.3d 1336, 1351, 94 USPQ2d 1161, 1172).

### EXAMPLES

Having now generally described this invention, the same will be better understood by reference to certain specific examples, which are included herein only to further illustrate the invention and are not intended to limit the scope of the invention as defined by the claims.

### Example 1

### Identification of Vaccine Candidate Proteins Using 2DGE

Subunit vaccine candidates for heterologous protection against Glaesserella parasuis were identified using 2D gel electrophoresis (2DGE) and immunoblotting.

To identify novel subunit vaccine candidates for heterologous protection against G. parasuis, previously generated serum from bacterin-vaccinated pigs that were protected (HS069 bacterin) or non-protected (Nagasaki bacterin) against heterologous challenge with 12939 was used to differentiate the antibody response using 2-D gel electrophoresis and immunoblotting.

Three virulent strains of G. parasuis were used here: 12939 (serovar 1), Nagasaki, and HS069 (serovar 5). G. parasuis 12939 is a SV1 strain originally isolated from the lung of a pig showing clinical signs of Glässer's disease, including pneumonia and polyserositis (Brockmeier S L, et al., 2014, “Virulence and draft genome sequence overview of multiple strains of the swine pathogen Haemophilus parasuis,” PLOS One 9 (8): e103787). Nagasaki is a SV5-type strain originally isolated from the meninges of a pig with septicemia and meningitis (Brockmeier S L, Supra). The SV5 strain, HS069, was isolated from the lung of a pig with respiratory disease associated with G. parasuis (Howell K J, et al., 2014, “The use of genome wide association methods to investigate pathogenicity, population structure and serovar in Haemophilus parasuis,” BMC Genomics 15:1179). All strains were grown on brain heart infusion (BHI) plates supplemented with 0.1 mg/mL nicotinamide adenine dinucleotide (NAD+) and 10% horse serum (BHI+).

Colostrum-deprived caesarian-derived (CDCD) pigs were obtained from Struve Labs (Struve Labs International; Manning, Iowa, USA). The National Animal Disease Center's Institutional Animal Care and Use Committee approved all animal experiments.

Sodium dodecyl sulfate-polyacrylamide gel electrophoresis (SDS-PAGE) was used to run proteins and whole cell sonicate (2 μg and 10 μg, respectively). Samples were reduced using NuPAGE™ LDS Sample Buffer (Thermo Fisher Scientific; Waltham, Massachusetts, USA) and 10× Bolt™ Sample Reducing Agent (Thermo Fisher Scientific). Samples were then loaded on WedgeWell™ 10% Tris-Glycine Gels (Thermo Fisher Scientific) and run for 90 minutes at 120 mV.

Samples were transferred to a nitrocellulose or polyvinylidene fluoride (PVDF) membrane using the iBlot™ Gel Transfer Stacks (Invitrogen; Carlsbad, California, USA). The membranes were blocked using StartingBlock™ (TBS) Blocking buffer (Thermo Fisher Scientific) for 30 minutes at room temperature. Primary (serum) and secondary antibody were diluted in 5% bovine serum albumin (BSA)/0.1% TWEEN™ 20 non-ionic surfactant in TBS. Blots were incubated with diluted swine serum (1:1000) overnight at 4° C. Membranes were washed with 0.1% TWEEN™ 20 non-ionic surfactant in TBS and incubated with diluted (1:10,000) goat anti-pig antibody labeled with horse radish peroxidase (goat anti-pig antibody-HRP; SeraCare; Milford, Massachusetts, USA) for 1 hour at room temperature. Membranes were washed and developed with SuperSignal™ West Dura Chemiluminescent Substrate (Thermo Fisher Scientific) or Pierce™ ECL Western Blotting Substrate (Thermo Fisher Scientific).

Enrichment for outer membrane proteins was accomplished using TRITON X-114 nonionic surfactant as previously described (Bordier C., 1981, “Phase separation of integral membrane proteins in Triton X-114 solution,” J. Biol. Chem. 256 (4): 1604-1607). In brief, 2 mL of in vitro-grown G. parasuis strain 12939 was resuspended in 2% v/v TRITON X-114 nonionic detergent (Sigma-Aldrich; St. Louis, Missouri, USA) in TE buffer (10 mM Tris-HCl, 1 mM Ethylenediaminetetraacetic Acid (EDTA)) pH 7.4 and rotated overnight at 4° C. Insoluble material was removed by centrifugation at 20,000×g for 30 minutes at 4° C. in a non-swinging bucket rotor. The supernatant was phase-separated by warming to 37° C. for 10 minutes followed by centrifugation at 13,000×g for 10 minutes. The aqueous phase (upper layer) was removed, and the detergent phase (lower layer) was washed with an equivalent amount of ice-cold TE buffer. Phase separation and washing of the detergent phase was repeated twice. Chloroform-methanol precipitation was used to remove detergent contaminants (Wessel D and Flugge U I, 1984, “A method for the quantitative recovery of protein in dilute solution in the presence of detergents and lipids,” Anal. Biochem. 138 (1): 141-143).

Samples were solubilized for two dimensional gel electrophoresis (2DGE) in 7 M urea, 2 M thiourea, 1% amidosulfobetaine-14 overnight at room temperature. Solubilized samples were prepared in 1 M DTT (GE Healthcare; Chicago, Illinois, USA), IPG buffer (GE Healthcare), and bromophenol blue and added to Immobiline™ DryStrips (GE Healthcare) to rehydrate overnight. Isoelectric focusing (IEF) was run on an Ettan IPGphor III (GE Healthcare) per the manufacturer's recommendations. After the first separation, strips were incubated in 1% DTT for 10 minutes, followed by 10 minutes in 2.5% iodacetamide (BIO-RAD, Hercules, CA). The second dimension was run using 7 cm, 12% Mini-PROTEAN™ TGX Stain-Free™ Gels (BIO-RAD).

The 24 cm gel and immunoblots were run as described above with minor modifications. The large gels were run in an Ettan™ DALTsix electrophoresis unit (GE Healthcare) overnight. Total protein was detected in 2-D gels with SYPRO™ Ruby protein gel stain (Thermo Fisher Scientific). Separated proteins were transferred to a glass plate for protein selection or PVDF membranes for protein visualization. The protein gels were bound to a glass plate with Bind-silane (GE Healthcare). Transfer of the 24 cm gels to PVDF membranes was done with the TE 77 PWR system (GE Healthcare). Membranes were incubated with serum and secondary antibody as described. Images were collected using the ChemiDoc™ MP Imaging System (BIO-RAD).

The trypsin digest was carried out using the In-Gel Tryptic Digestion Kit (Thermo Fisher Scientific) per the manufacturer's recommendations. Pierce™ C18 Spin Columns (Thermo Fisher Scientific) were used for isolation and clean-up of blot-isolated proteins. The clean-up protocol was followed as recommended with modifications: equilibration and wash solutions used were 0.1% formic acid/5% acetonitrile (ACN); sample buffer 0.4% formic acid/20% ACN; elution buffer 0.1% formic acid/95% ACN.

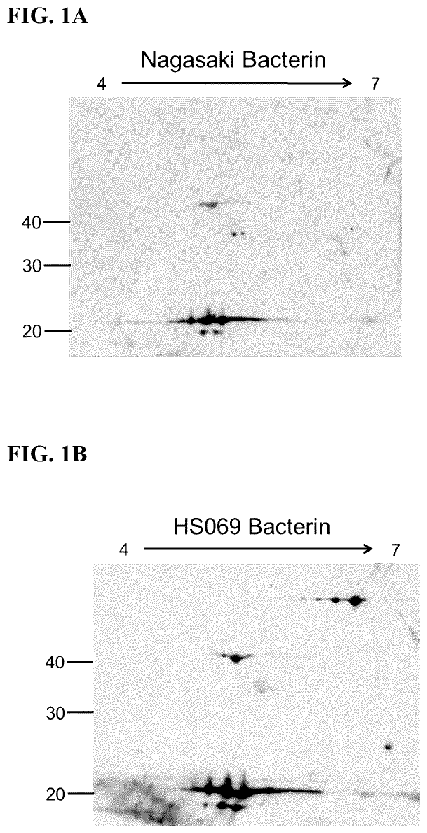

To elucidate proteins of interest from the detergent phase, 2DGE was employed to gain better separation of proteins and enable identification. The detergent phase containing OMPs was run on 2-D gels ranging from a pH of 4 to 7 and 3 to 10. Most outer membrane proteins (OMPs) were observed within a pH of 4 to 7 and gels of pH 4 to 7 were used for the remainder of the experiments. Two-dimensional gels were transferred to immunoblots and probed with serum obtained 42 dpv from animals vaccinated with HS069 bacterin (protected) or Nagasaki bacterin (non-protected). As seen in FIG. 1A and FIG. 1B, proteins bound by serum antibodies from protected animals were greater in number and distinct from those bound by serum antibodies from non-protected animals.

### Example 2

### Protein Search and Identification

Subunit vaccine candidates were identified by performing mass spectrometry on proteins with differential representation between blots probed with serum from HS069- or Nagasaki-vaccinated animals.

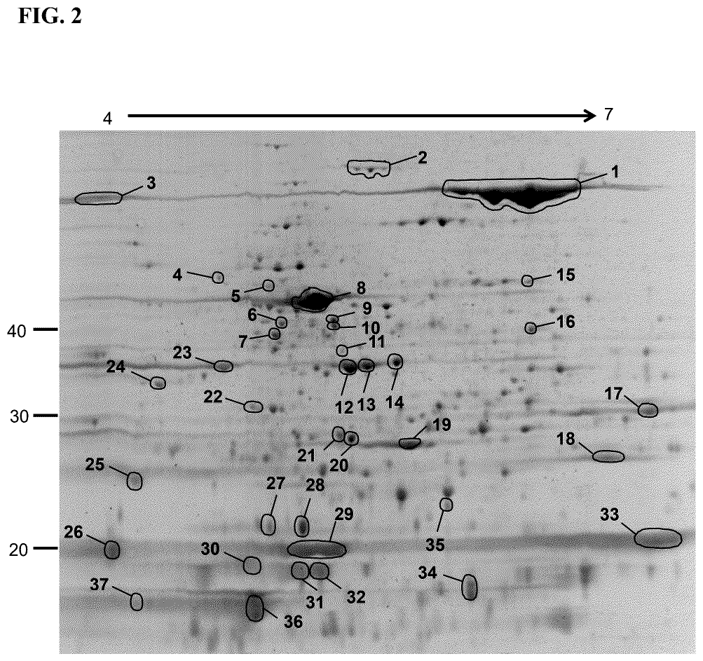

Proteins of interest, both similarly and differentially bound by hyperimmune serum, were excised from the 2DGE. FIG. 2 depicts a photograph of a two dimensional electrophoresis gel (2DGE) stained with SYPRO Ruby protein gel stain for visualization of outer membrane proteins present. Proteins identified with a 1; 2; 3; 4; 5; 6; 7; 9; 10; 11; 15; 16; 17; 18; 19; 20; 21; 22; 35; or 36 correlate to those identified in the protected immunoblot only, while proteins identified with a 8; 12; 13; 14; 23; 24; 25; 26; 27; 28; 29; 30; 31; 32; 33; 34; or 37 were proteins similar between the protected and non-protected groups. The 40 proteins were identified by mass spectrometry. The proteins identified with a 1; 2; 3; 4; 5; 6; 7; 9; 10; 11; 15; 16; 17; 18; 19; 20; 21; 22; 35; or 36, specific to the protected immune response, were thought to have the highest likelihood of being involved in heterologous protection. The predicted identity of the proteins of interest listed in Table 2A and Table 2B (below) were determined based on the number of unique peptides observed by MS, molecular weight aligning with size observed in gel, and protein location/function indicative of an OMP.

Samples were injected onto an HPLC column using a Proxeon Easy-nLC (Thermo Fisher Scientific) connected to the mass spectrometer. Chromatography was completed using a trapping column (Proxeon Easy-Column, 2 cm, ID 100 μm, 5 um, 120A, C18) and an analytical column (Proxeon Easy-Column, 10 cm, ID 75 μm, 3 um, 120A, C18). The gradient was composed of Mobile Phase A (95% H₂O: 5% acetonitrile, and 0.1% formic acid) and Mobile Phase B (5% H₂O: 95% acetonitrile, and 0.1% formic acid) and was as follows: 0% B for 2 minutes, 0 to 5% B in 1 minute, 5 to 18% B in 20 minutes, 18 to 30% B in 2 minutes, 30 to 90% B in 1 minute, and then held at 90% B for 10 minutes at a flow rate throughout the gradient of 300 nL per minute. The analytical column was connected to a PicoTip™ Emitter (New Objectives, Woburn, Massachusetts, USA) cut to size. The column and Emitter were attached to a LTQ OrbiTrap™ Velos Pro (Thermo Fisher Scientific) mass spectrometer using the Proxeon Nanospray Flex Ion Source. The capillary temperature was set at 275° C. and spray voltage was 2.8 kV. The mass spectrometer used a data dependent method. In MS mode, the instrument was set to scan 300 to 2,000 m/z with a resolution of 30,000 FWHM. A minimal signal of 20,000 could trigger MS/MS, and ten consecutive MS/MS were possible. The activation type used was CID, and repeat mass exclusion was set to 120 seconds.

Mass spectrometry data from all gel spots were analyzed using Protein Discoverer, Version 2.1 (Thermo Fisher Scientific). Sequest™ and Amanda™ search engines were set up to explore the UniProt™ proteome database for Glaesserella parasuis proteins (Organism ID 738; downloaded on Nov. 3, 2016). The search engines were used with a fragment ion mass tolerance of 1.2 Da and a parent ion tolerance of 10.0 ppm. Carbamidomethyl of cysteine, acetylation of the N-terminus and lysine, formylation of lysine, oxidation of methionine, and phosphorylation of serine, threonine, and tyrosine were specified as variable modifications. High peptide confidence was used. Listed in Table 2A and Table 2B below are the identities of the predicted proteins of interest, indicating the spot number, the UniProt Accession number, the description found in UniProt, the coverage, the number of unique peptides, the molecular weight in kDa, the Sequest™ HT score, and the MS Amanda score.

TABLE 2A
PREDICTED IDENTITY OF PROTEINS OF INTEREST

| Spot # | Accession | Description | Coverage |
|---|---|---|---|
| 1 | 633282331 | outer membrane protein assembly protein YaeT | 70.93167702 |
| 2 | 633282331 | outer membrane protein assembly protein YaeT | 65.96273292 |
| 2 | 513127695 | pyruvate dehydrogenase subunit E1 | 37.74011299 |
| 3 | 633282331 | outer membrane protein assembly protein YaeT | 68.44720497 |
| 4 | 633282331 | outer membrane protein assembly protein YaeT | 54.16149068 |
| 5 | 666684705 | Omp85 | 30.65512979 |
| 5 | 544680530 | phosphomannomutase | 41.12318841 |
| 6 | 498484049 | UDP-N-acetylmuramoyl-L-alanyl-D-glutamate--2,6- | 37.7016129 |
| | | diaminopimelate ligase (murE) | |
| 6 | 666684705 | Omp85 | 17.92336218 |
| 7 | 666684705 | Omp85 | 32.5092707 |
| 9 | 544681590 | penicillin-binding protein activator LpoA | 59.24006908 |
| 9 | 666684705 | Omp85 | 33.00370828 |
| 10 | 544681590 | penicillin-binding protein activator LpoA | 54.23143351 |
| 10 | 666684705 | Omp85 | 38.07169345 |
| 11 | 544681590 | penicillin-binding protein activator LpoA | 38.16925734 |
| 11 | 666684705 | Omp85 | 32.26205192 |
| 15 | 666684705 | Omp85 | 26.45241038 |
| 15 | 544681590 | penicillin-binding protein activator LpoA | 23.66148532 |
| 16 | 498484463 | NADH:ubiquinone reductase (Na(+)-transporting) | 40.625 |
| | | subunit A | |
| 17 | 460513573 | D-alanyl-D-alanine carboxypeptidase fraction A | 43.76590331 |
| 18 | 529262359 | Outer membrane lipoprotein A | 67.73162939 |
| 18 | 529265750 | Outer membrane lipoprotein A | 19.93569132 |
| 19 | 1016079623 | Thiamine biosynthesis protein ApbE | 34 |
| 20 | 1016079623 | Thiamine biosynthesis protein ApbE | 18 |
| 21 | 1016079623 | Thiamine biosynthesis protein ApbE | 14.57142857 |
| 22 | 476407734 | Phosphoglycerate kinase | 11.25319693 |
| 35 | 544681431 | 4-hydroxy-tetrahydrodipicolinate synthase | 31.86440678 |
| 36 | 1016079789 | ABC transporter substrate-binding protein | 12.91512915 |

TABLE 2B
PREDICTED IDENTITY OF PROTEINS OF INTEREST

| Spot | | | | Score MS |
|---|---|---|---|---|
| # | # Unique Peptides | MW [kDa] | Score Sequest HT | Amanda |
| 1 | 64 | 90.232 | 1474.151308 | 74978.2372 |
| 2 | 49 | 90.232 | 2592.755023 | 125603.1374 |
| 2 | 37 | 98.778 | 204.9492233 | 13597.83973 |
| 3 | 59 | 90.232 | 754.1437316 | 41658.33401 |
| 4 | 40 | 90.232 | 394.4162972 | 20087.9579 |
| 5 | 19 | 90.74 | 106.7312157 | 5217.015474 |
| 5 | 20 | 60.003 | 64.48337913 | 3673.753021 |
| 6 | 17 | 54.695 | 80.15718389 | 4626.826224 |
| 6 | 12 | 90.74 | 34.27835274 | 1999.386489 |
| 7 | 22 | 90.74 | 116.6251886 | 4799.822735 |
| 9 | 29 | 64.111 | 659.5503209 | 30399.89489 |
| 9 | 21 | 90.74 | 98.29631805 | 4969.99658 |
| 10 | 25 | 64.111 | 368.0266004 | 15200.34886 |
| 10 | 23 | 90.74 | 140.0894084 | 6450.031367 |
| 11 | 19 | 64.111 | 158.916729 | 6701.839573 |
| 11 | 20 | 90.74 | 135.3641214 | 6050.068535 |
| 15 | 15 | 90.74 | 83.18866229 | 3859.522111 |
| 15 | 10 | 64.111 | 58.25997925 | 2261.608411 |
| 16 | 21 | 48.483 | 92.5118258 | 4939.651159 |
| 17 | 15 | 43.205 | 101.8467631 | 5427.079974 |
| 18 | 15 | 34.612 | 146.6876192 | 7923.857918 |
| 18 | 1 | 34.001 | 37.79742265 | 2221.088092 |
| 19 | 12 | 38.824 | 159.3401833 | 9073.988175 |
| 20 | 5 | 38.824 | 22.88975215 | 2071.463289 |
| 21 | 4 | 38.824 | 10.9923923 | 645.9476399 |
| 22 | 4 | 41.375 | 7.233653069 | 294.9441747 |
| 35 | 7 | 31.439 | 37.38512492 | 1634.332264 |
| 36 | 3 | 29.127 | 4.201957226 | 332.5555166 |

Each spot does not necessarily represent one individual protein-some spots represent more than one protein separating together, and some proteins separate into multiple spots (YaeT for example). Additionally, fragments are detected, and the fragments may hit to multiple proteins. Some proteins are more likely due to a greater coverage (more fragments of that protein were identified).

The three most prevalent proteins (ApbE, LpoA, and YaeT) were selected for evaluation as a subunit vaccine. The nucleotide sequence of apbE, IpoA, and yaeT were compared using Geneious™ 9.0.5 (Biomatters Ltd., Auckland, New Zealand) across twelve (12) G. parasuis isolates representing nine (9) serotypes: HS069 (ERS132117), SH0165 (CP001321), Nagasaki (APBT00000000), SW114 (APBU00000000), MN-H (APBV0000000), 12939 (APBW00000000), 29755 (ABKM00000000), 84-15995 (APBX00000000), H₄₆₅ (APBY00000000), D74 (ABPZ00000000), 174 (APCA00000000), and SW140 (APCB00000000). The genes were extracted from the genome and compared using multiple sequence alignments.

To further confirm the cross protective proteins identified by mass spectrometry, a cross-blotting experiment was utilized. That is, Nagasaki OMPs and HS069 OMPs were run on 2DGE and immunoblots probed with serum from protected animals given HS069 bacterin and non-protected animals given Nagasaki bacterin. The resulting blots showed similar patterns of spots within a treatment group (Nagasaki serum vs HS069 serum). For instance, protein spots corresponding to YaeT did not appear in any of the blots when non-protected serum was used, but were present in all blots with protected serum.

Among the twelve (12) isolates screened, the three proteins selected were highly conserved. The apbE gene was found in all screened genomes and sequence identity was >99% between all isolates. The IpoA gene was found in eleven (11) of the genomes with sequence identity >93% for all isolates. Finally, yaeT was present in ten (10) of the 12 genomes screened and identity conservation was >98% in those isolates. Though some isolates appeared to lack IpoA or yaeT, the genomes utilized in this screen were drafts and the gene of interest may have been absent due to the location of gaps within the draft genomes.

This Example shows that using 2DGE, immunoblotting, and mass spectrometry, apbE, IpoA, and yaeT were identified as subunit vaccine candidates.

### Example 3

### Vaccine Study with rApbE, rLpoA, and rYaeT

Recombinant proteins (rApbE, rLpoA, rYaeT) were generated by GenScript (GenScript, Piscataway, NJ) using the coding sequence from G. parasuis HS069, and used in a vaccine study. The coding sequence of apbE, IpoA, and yaeT were individually cloned into the pUC57 vector. Each gene was then subcloned into the pET30a vector and a histidine tag was added for protein purification. The proteins were expressed in Escherichia coli BL21 Star (DE3). Proteins were purified from cell pellets using a nickel column and sterilized by filtration (0.22 μm filter). Protein concentration was determined by Bradford protein assay using bovine serum albumin as a standard. Protein purity and molecular weight were determined by SDS-PAGE and western blot confirmation.

Pigs were vaccinated on day 0 and 21 of the experiment with one of three treatments: subunit vaccinated (Combination 3: rApbE, rLpoA, rYaeT), bacterin vaccinated, or sham vaccinated. Subunit vaccinated animals were inoculated intramuscularly with 2 mL containing 100 μg of each protein (rApbE, rLpoA, and rYaeT) and 20% Emulsigen D (MVP Laboratories; Omaha, Nebraska, USA). Bacterin-vaccinated animals were inoculated intramuscularly with 2 mL containing 1×10⁹ colony forming units (CFU) of formalin-inactivated G. parasuis HS069 and 20% Emulsigen D. Sham-vaccinated animals received intramuscular injections of 2 mL PBS with 20% Emulsigen D. Animals were challenged intranasally on day 43 of the experiment with either the homologous G. parasuis strain (HS069) or a heterologous G. parasuis strain (12939) at approximately 1×10⁸ CFU/mL. Post-challenge, pigs were monitored for clinical signs of Glässer's disease (lethargy, depression, lameness, respiratory distress, and neurologic signs). Animals presenting with severe systemic disease were euthanized and samples were collected. Surviving pigs were euthanized 15 days after challenge (day 58). At necropsy, nasal swab, serum, serosal swab, joint fluid, cerebrospinal fluid (CSF), and lung lavage were collected and plated on BHI+ agar for enumeration.

Blood was collected on day 0, day 21, and day 43 in BD Vacutainer™ serum separator tubes (SST) (Becton Dickinson; Franklin Lakes, New Jersey, USA) and serum was stored at −80° C. until enzyme linked immunosorbent assays (ELISAs) were run. Immulon™-2 plates were coated overnight with 100 μL per well of 0.5 μg/mL sonicated G. parasuis or 0.5 μg/mL recombinant protein in 100 mM carbonate-bicarbonate buffer (pH 9.6). After washing three times with 1×PBS with 0.05% Tween™ 20 nonionic surfactant (PBST), plates were blocked for 2 hours with 200 μL of 2% bovine serum albumin (BSA) in PBST. Plates were washed three times with PBST and probed with 100 μL of two-fold serial dilutions of swine anti-sera in 1% BSA in PBST for 2 hours. All dilutions were run in duplicate. After washing with PBST, IgG recognizing the recombinant proteins was incubated with 100 μL of goat anti-swine IgG conjugated with horse radish peroxidase (SeraCare; Milford, Massachusetts, USA) at a 1:20,000 dilution in 1% BSA in PBST for 1 hour. Plates were washed with PBST and 100 μL of tetramethylbenzidine (TMB) substrate (Life Technologies; Carlsbad, California, USA) was added to each well for detection. The reaction was stopped after 5 minutes with 50 μL of 2N H₂SO₄ and the optical density at 450 nm was measured with correction at 655 nm. GraphPad Prism™ software (GraphPad Software; La Jolla, California, USA) was utilized to model the data as a nonlinear function of the log₁₀ dilution and the log (agonist)-versus response variable slope four-parameter logistic model. Gnotobiotic pig serum was used to dictate endpoint titers, with two times the average gnotobiotic serum sample as the cutoff.

Peripheral blood mononuclear cells (PBMCs) were isolated from blood samples taken on day 21 and day 43. Blood was collected in BD Vacutainer™ cell preparation tubes (CPT) (Becton Dickinson) and PBMCs were isolated as described by Braucher D R, et al. (2012, “Intranasal vaccination with replication-defective adenovirus type 5 encoding influenza virus hemagglutinin elicits protective immunity to homologous challenge and partial protection to heterologous challenge in pigs,” Clin. Vaccine Immunol. 19 (11): 1722-1729). Briefly, following centrifugation, the buffy coat was collected. Cells were washed with PBS, pelleted, and resuspended in PBS. PBMCs were filtered with a 40 μm screen filter, washed with PBS, and pelleted. Cells were resuspended in RPMI media, and the cell concentration and viability were assessed with the Muse instrument (Cytek Biosciences, Fremont, California, USA). PBMCs were seeded onto IFN-γ enzyme-linked immunosorbent spot (ELISpot) plates (R&D Systems, Minneapolis, Minnesota, USA) at 2.5×10⁵ cells per well. PBMCs were stimulated in duplicate wells with each recombinant protein (1 μg/mL) and G. parasuis HS069 and 12939 (0.16 μg/mL) in a total volume of 250 μL. Control wells were treated with medium alone or pokeweed mitogen (0.25 μg/mL) as a negative and positive control, respectively. After 18 hours of stimulation, the assay was completed as per manufacturer's recommendations. When plates were dry, an SUV ImmunoSpot™ instrument and software (Cellular Technology Ltd., Shaker Heights, Ohio, USA) were used to calculate total spots corresponding to IFN-γ secreting cells for each treatment. Treatment averages were calculated for each group using the average IFN-γ secreting cells for each pig calculated from the duplicate wells.

Pooled sera from subunit-, bacterin-, and sham-vaccinated animals was evaluated for antibody-mediated complement killing as previously described by Brockmeier S L, et al. (2013, “Virulence, transmission, and heterologous protection of four isolates of Haemophilus parasuis,” Clin. Vaccine Immunol. 20 (9): 1466-1472). Briefly, for each serum sample, 20 μL of HI swine serum was combined with 70 μL of non-HI guinea pig serum and 10 μL of G. parasuis suspension at OD₆₀₀ of 0.42 (˜106 CFU). Following incubation at 37° C. for 1 hour, serial dilutions of each sample were plated on BHI+ agar for enumeration. Pooled sera from day 43 was heat-inactivated (HI) by heating to 56° C. for 1 hour and utilized as the antibody source. Guinea pig serum was utilized as a source of complement. Serum was tested with G. parasuis strains HS069 and 12939.

Receptor-mediated phagocytosis was assessed using G. parasuis 12939. Plate-grown bacteria were suspended in PBS at an OD₆₀₀ around 0.42 (approximately 1×10⁸ CFU/mL). In 96-well round bottom plates, 50 μL of heparinized swine blood was combined with 20 μL of pooled HI serum and 30 μL of bacterial suspension. Swine serum was from one of the treatment groups (subunit-, bacterin-, or sham-vaccinated) or, as a control, was from day 0 (DO) of the study prior to vaccination. The plates were incubated for 1 hour at 37° C. with agitation. After incubation, serial dilutions were plated, and CFU/mL was calculated for comparison.

Statistics were performed in GraphPad Prism™ 7 (GraphPad, La Jolla, California, USA). Analysis of survival was completed using the Kaplan and Meier method and comparisons of survival curves utilized the log rank test. Group comparisons were completed using one-way ANOVA for log₁₀ antibody titers, ELISpot data, cytokine production data, complement killing, and whole blood killing. A P value of <0.05 was used as the cutoff to denote statistical significance.

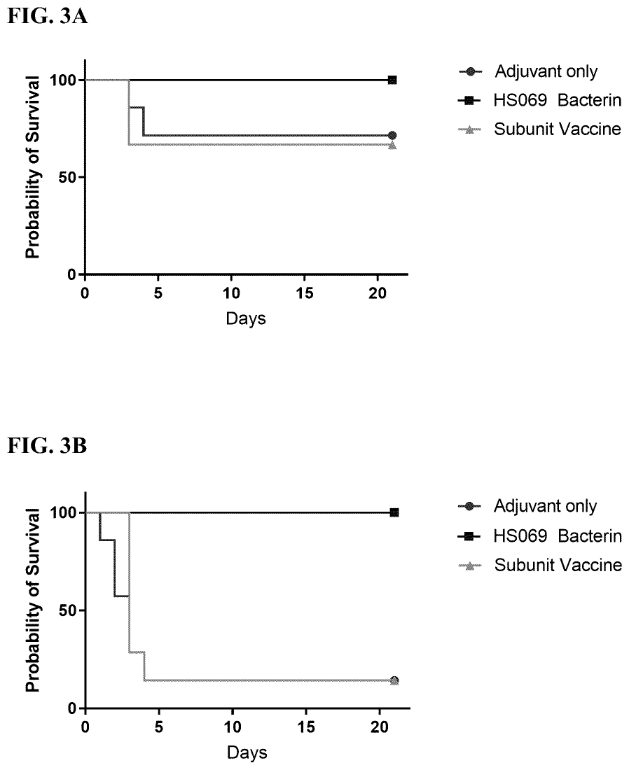

Survival curves after homologous challenge are shown in FIG. 3A and after heterologous challenge are shown in FIG. 3B. As seen in FIG. 3A, challenge with G. parasuis HS069 (homologous) caused 29% mortality in the control group, which was atypical of challenge with this strain. The subunit vaccinated group had a 33% mortality with the homologous challenge, which was not statistically different than that of the control animals. No mortality was seen in bacterin vaccinated animals, which was consistent with previous vaccination studies; however, because challenge did not produce mortality typical of HS069, this was not statistically significant (p=0.14).

As seen in FIG. 3B, challenge with the heterologous strain, G. parasuis 12939, resulted in severe systemic signs in 86% of sham (6/7) and subunit (Combination 3) vaccinated animals (6/7). Similar to the previous study, none of the G. parasuis HS069 bacterin-vaccinated animals succumbed to heterologous challenge with G. parasuis 12939 (p=0.001).

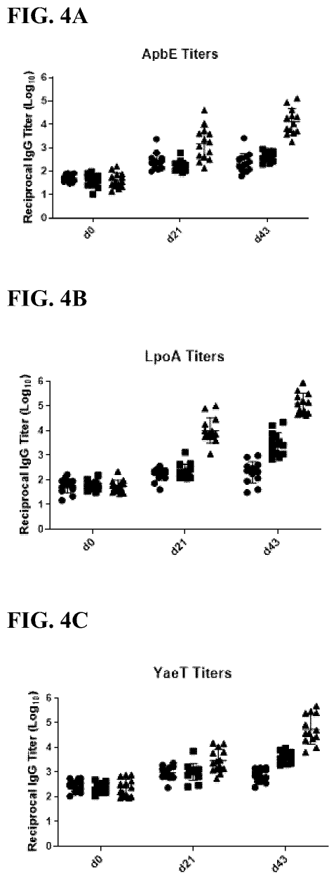

Antibody titers to rApbE, rLpoA, and rYaeT are visualized in FIG. 4A to FIG. 4C. Subunit vaccination induced a protein-specific response to rApbE (FIG. 4A), rLpoA (FIG. 4B), and rYaeT (FIG. 4C). This response was significantly greater than the one seen in sham vaccinated controls. An amnestic response was seen to the boost vaccination. The bacterin vaccinated animals also showed elevated titers to rLpoA and rYaeT when compared to the sham vaccinated animals on day 43 (p<0.001); however, this was significantly lower than that of the subunit vaccinated animals (p<0.001).

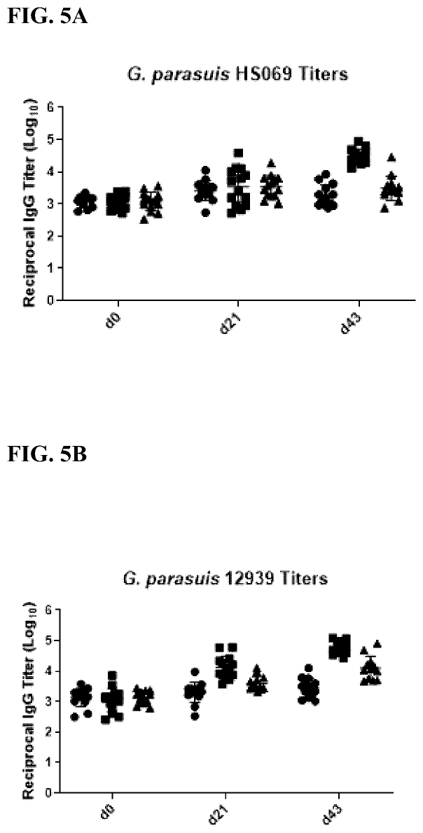

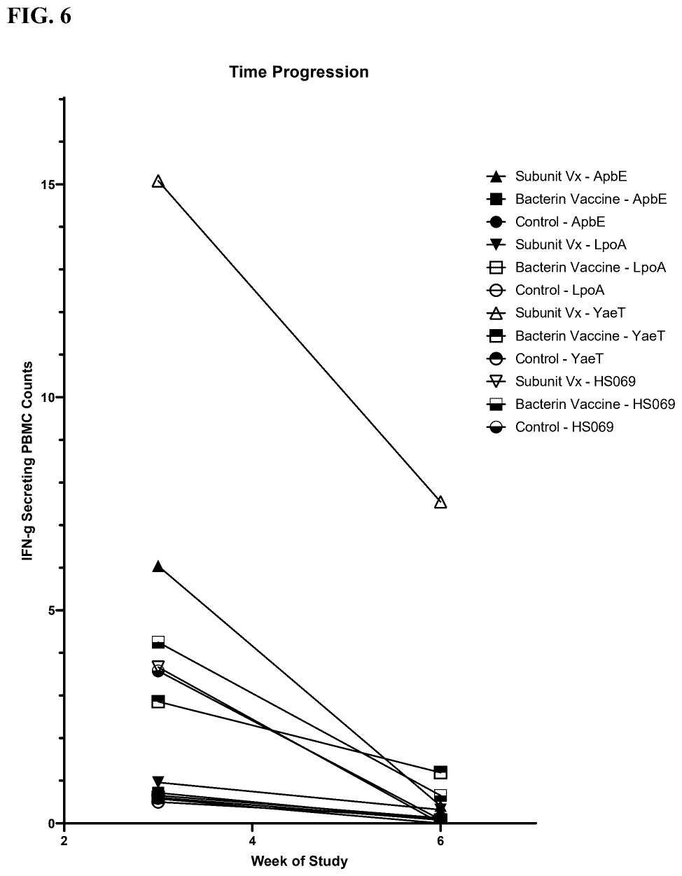

Titers to the G. parasuis challenge strains are shown in FIG. 5A and FIG. 5B. As seen in FIG. 5A, subunit-vaccinated (Composition 3) and sham-vaccinated animals had no statistical difference in antibody titers to G. parasuis HS069 (p=0.14). However, as seen in FIG. 5B, there was an elevation in the titers of subunit (Combination 3) vaccinated animals to G. parasuis 12939 when compared with sham vaccinated controls (p<0.001). The titers of subunit-vaccinated animals were significantly lower than that of bacterin-vaccinated animals for both G. parasuis HS069 and 12939 (p<0.001).

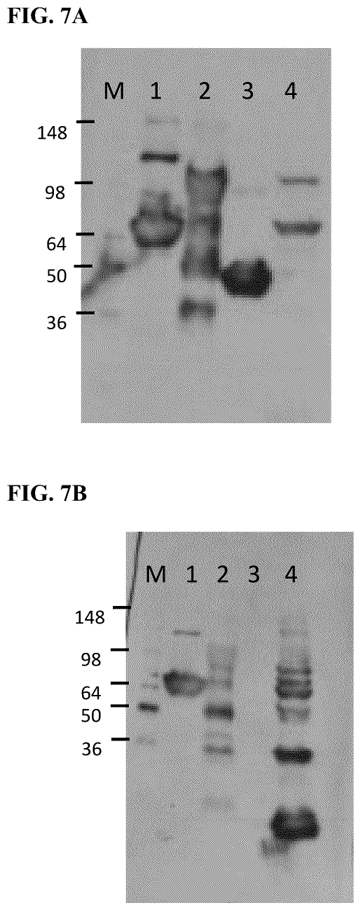

As seen in FIG. 7, western blotting revealed cross-reactivity between pooled sera from subunit-vaccinated animals and G. parasuis HS069 whole cell sonicate. Additionally, pooled sera from bacterin-vaccinated animals cross reacted with rLpoA and rYaeT. This indicated expression of IpoA and yaeT′ by G. parasuis in vitro and showed reactivity of the antibody generated from subunit vaccination with G. parasuis.

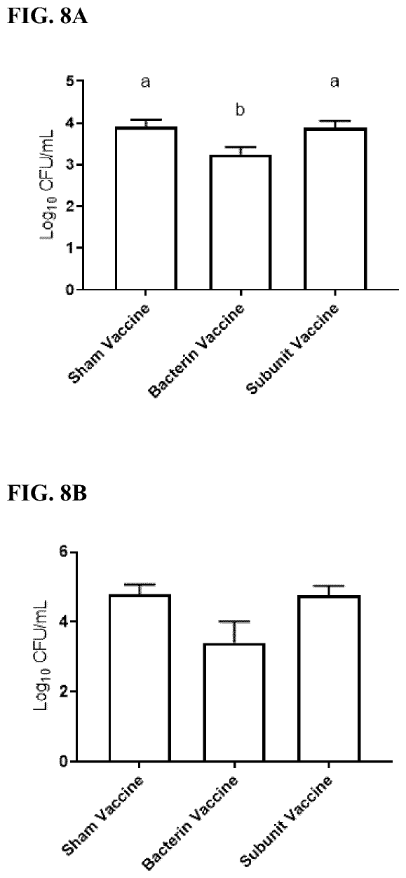

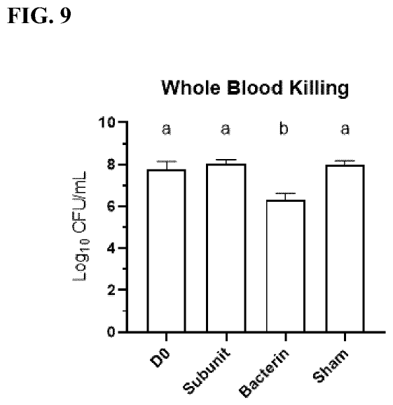

Antibody function was assessed using complement-mediated lysis and receptor-mediated phagocytosis assays. As seen in FIG. 8A, complement-mediated lysis was significantly more effective against G. parasuis HS069 when enhanced by HI serum from bacterin-vaccinated animals than sham-vaccinated (p=0.0273) or subunit (Combination 3)-vaccinated animals (p=0.0477). This trend also appeared with G. parasuis 12939; however, as seen in FIG. 8B, it did not reach statistical significance (p=0.0738 and 0.0835 for sham and subunit vaccinated animals, respectively). As seen in FIG. 9, assessment of opsonization and phagocytosis by whole blood killing revealed a greater reduction in log₁₀ CFU/mL when blood was supplemented with pooled, HI serum from bacterin-vaccinated animals than subunit (Combination 3) (p<0.01) and sham vaccinated animals (p<0.01).

### Example 4

### Immunoprecipitation Methods

Immunogenic proteins were identified through immunoprecipitation using serum with known potential to provide heterologous protection. From the identified proteins, three (catalase, PBP5, and PotA) were selected and evaluated as vaccine antigens to protect pigs against G. parasuis disease.

Immunoprecipitation was done in three iterations: (1) preclearing with DO serum from Nagasaki-vaccinated animals and probing with D42 serum from Nagasaki-vaccinated animals, (2) preclearing with DO serum from HS069-vaccinated animals and probing with D42 serum from HS069-vaccinated animals, and (3) preclearing with D42 serum from Nagasaki-vaccinated animals and probing with D42 serum from HS069-vaccinated animals. Whole cell sonicate (10 μg) was precleared in a 500 μL volume containing 10 μL serum, 50 μL Pierce™ Protein A Magnetic Beads (ThermoFisher Scientific), and 5 μL protease inhibitor cocktail (ThermoFisher Scientific) containing AESBF HCl (hydrogen 4-(2-aminoethyl)benzene-1-sulfonyl fluoride chloride), Aprotinin, E-64, EDTA, and Leupeptin in PBS. The sonicate was precleared by rocking for 1 hour at room temperature with the indicated preclearing serum. The supernatant was harvested and combined with 50 μL magnetic beads and 10 μL probing serum. Precleared sonicate was probed by rocking at room temperature for 1 hour with the probing serum as indicated. After probing, bead-bound antibodies were washed with phosphate buffered saline (PBS) three times. Bound protein was then eluted from the beads by adding 50 μL 2M glycine and agitating for 5 minutes at room temperature. Elution was repeated and then neutralized by adding an equal volume of 1M Tris-HCl. The elution was mixed with 800 μL ice-cold acetone and incubated at −20° C. for 1 hour. The mixture was centrifuged at 15000×G for 10 minutes to pellet proteins. The supernatant was decanted, and the pellet was air dried. When dry, the pellet was resuspended in PBS. Proteins were separated by SDS-PAGE. Bands of interest were excised and submitted to Iowa State University's Protein Facility for identification by mass spectrometry.

Proteins identified by Iowa State University's Protein Facility were screened by score and cell compartment. Proteins with a score over 50 were included for further analysis. A list of identified proteins was generated for each IP iteration. Lists were compared to identify proteins unique to or over-represented in the protective immune response (HS069-vaccinated). Proteins were then further evaluated for cellular localization, known virulence potential, or previous assessment for vaccine potential in related bacterial species. Finally, three proteins which were thought to have good potential as vaccine immunogens (catalase, PBP5, and PotA) were selected and produced by GenScript Biotech by cloning and expression as described in Example 3.

The nucleotide and amino acid identity of catalase, PBP5, and PotA were compared across 11 G. parasuis isolates representing 9 serovars using Geneious™ Prime (Biomatters Ltd.): SH0165 (CP001321), Nagasaki (APBT00000000), SW114 (APBU00000000), MN-H (APBV0000000), 12939 (APBW00000000), 29755 (ABKM00000000), 84-15995 (APBX00000000), H₄₆₅ (APBY00000000), D74 (ABPZ00000000), 174 (APCA00000000), and SW140 (APCB00000000). The extracted genes and translated proteins were compared using multiple sequence alignments.

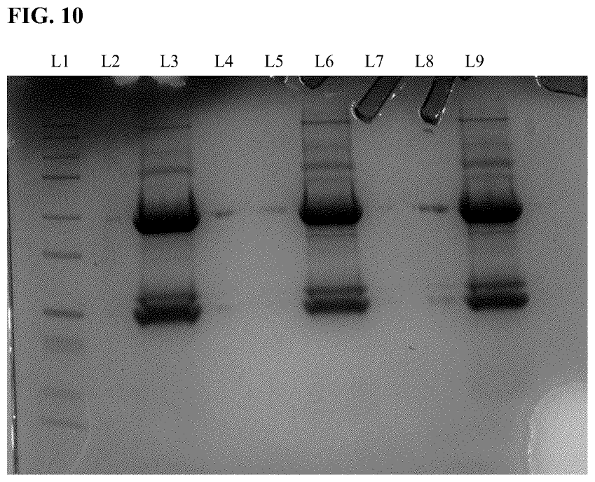

Proteins were isolated by immunoprecipitation and separated by SDS-PAGE. FIG. 10 depicts an image of an SDS-PAGE where lane 1 (L1) contains protein markers, lane 3 (L3) contains immunoprecipitated proteins isolated by preclearing with Nagasaki-vaccinated DO serum and probing with Nagasaki-vaccinated D42 serum (iteration 1); lane 6 (L6) contains immunoprecipitated proteins isolated by preclearing with HS069-vaccinated DO serum and probing with HS069-vaccinated D42 serum (iteration 2); lane 9 contains immunoprecipitated proteins isolated by preclearing with Nagasaki-vaccinated D42 serum and probing with HS069-vaccinated D42 serum (iteration 3), lanes not listed were run empty. Bands were excised and sequenced by mass spectrometry. The number of bands sent for sequencing were as follows for each iteration: (1) 5 bands, (2) 6 bands, and (3) 7 bands.

From these, three proteins with potential as vaccine candidates were selected: catalase, PotA-spermidine/putrescene ABC transporter ATPase protein, PBP5-D-alanyl-D-alanine carboxypeptidase fraction A. The three selected proteins were highly conserved in the 11 screened G. parasuis isolates. Catalase was found in 10 of the genomes and it had >96% nucleotide identity and >98% amino acid identity. PBP5 was found in only 6 of the genomes. It had >96% nucleotide identity and >98% amino acid identity. PotA was found in 10 of the genomes and it had >98% nucleotide identity and >99% amino acid identity.

### Example 5

### Vaccine Study with Catalase, PBP5, and PotA

Recombinant proteins of catalase, PBP5, and PotA (identified above) were produced by GenScript Biotech and used in a vaccine study.

All animal studies were approved by the National Animal Disease Center's Institutional Animal Care and Use Committee. Sixteen cesarean derived, colostrum deprived (CDCD) pigs derived at the National Animal Disease Center were distributed into two groups. Group 1 was vaccinated with a protein vaccine containing 100 μg of each protein (Combination 4: catalase, PBP5, and PotA) in PBS with 20% Emulsigen™-D (MVP Laboratories Inc). Group 2 was vaccinated with a sham vaccine containing PBS with 20% Emulsigen™-D. Animals were vaccinated on DO and D21 and challenged intranasally on D56 with 2 mL of 1×10⁸ CFU/mL G. parasuis HS069. Following challenge, animals were evaluated twice daily for clinical signs of G. parasuis. If clinical signs were present but not severe, a third evaluation was completed approximately 5 hours later. Surviving animals were euthanized 14 days post-challenge. At necropsy, the following samples were collected for bacteriologic evaluation: nasal swab, serosal swab, joint fluid, cerebrospinal fluid (CSF), serum, and lung lavage. Serum was collected on DO, D21, and D42 and stored at −80° C. until serum antibody assessment.

Serum antibody was assessed using enzyme-linked immunosorbent assays (ELISAs). Plates were coated overnight with 0.5 μg/mL of G. parasuis HS069 or 0.5 μg/mL of recombinant protein Combination 4 (catalase, PBP5, or PotA) in 100 mM carbonate-bicarbonate buffer. Plates were blocked with 2% bovine serum albumin (BSA) in 1×PBS with 0.05% Tween™ 20 (PBST) and probed with two-fold serial dilutions of swine serum in 1% BSA in PBST. Bound antibody was detected with a 1:20,000 dilution of horse radish peroxidase conjugated goat anti-swine IgG in 1% BSA in PBST. Plates were developed with tetramethylbenzidine, and the reaction was stopped with 2N H₂SO₄. The optical density was read at 450 nm and the data was modeled in GraphPad Prism™ (GraphPad Software) to predict the endpoint titer using two times the average gnotobiotic pig serum as the cutoff.

Statistical analysis was run in GraphPad Prism™. Serum antibody titers were compared using a two-way ANOVA. Survival was assessed using the Kaplan and Meier method and curve comparisons with the log-rank test. Results were considered statistically significant at P<0.05.

One vaccinated animal developed clinical signs including lethargy, tremors, and fever after the second vaccine dose and was euthanized. No other animals displayed any adverse effects following vaccination.

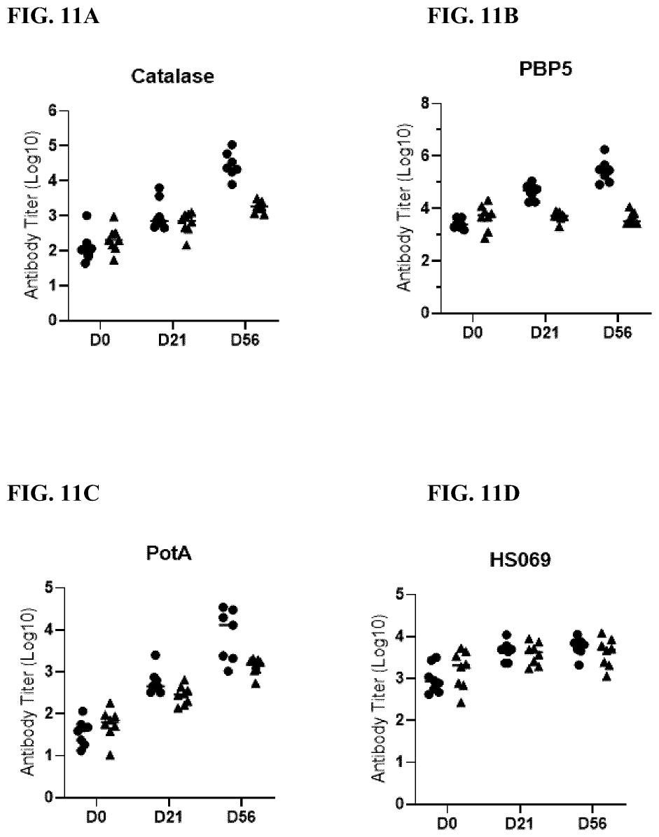

The serum antibody response to vaccine antigens from Combination 4 (catalase, PBP5, and PotA) and HS069 evaluated by ELISA are shown in FIG. 10A to FIG. 10D. As seen in FIG. 11A, FIG. 11B, and FIG. 11C, statistical rise in titer was seen for the vaccine antigens on D56 of the study (P<0.01). FIG. 11D shows that there was no rise in titer to HS069 for animals vaccinated with Combination 4 (P=0.80).

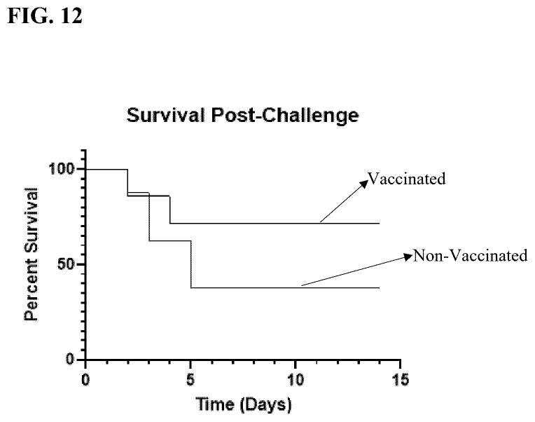

FIG. 12 shows the percent animal survival post challenge. More animals vaccinated with Combination 4 survived post-challenge (5/7) than control animals (3/8); however, there was no statistical difference in survival (P=0.31).

This Example shows that a subunit vaccine prepared with recombinant catalase, PBP5, and PotA (Combination 4) may provide some protection when compared with control.

### Example 6

### Vaccine Studies with Lipoprotein A Combinations

The effects of a subunit vaccine comprising recombinant LpoA, Lipoprotein A (LppA), and YaeT (Combination 1) or catalase, LppA, and PBP5 (Combination 2) were studied.

Pigs were vaccinated with a protein vaccine containing 100 μg of each protein (Combination 1: LppA, YaeT, and catalase) or (Combination 2: LppA, PBP5, and catalase) in PBS with 20% Emulsigen™-D (MVP Laboratories Inc). or with a sham vaccine containing PBS with 20% Emulsigen™-D. Animals were vaccinated on DO and D21 and challenged intranasally on D56 with 2 mL of 1×10⁸ CFU/mL G. parasuis HS069. Following challenge, animals were evaluated twice daily for clinical signs of G. parasuis. If clinical signs were present but not severe, a third evaluation was completed approximately 5 hours later. Surviving animals were euthanized 14 days post-challenge. At necropsy, the following samples were collected for bacteriologic evaluation: nasal swab, serosal swab, joint fluid, cerebrospinal fluid (CSF), serum, and lung lavage. Serum was collected on DO, D21, and D42 and stored at −80° C. until serum antibody assessment.

Serum antibody was assessed using ELISAs to test for each individual protein as described in Example 5. Plates were coated overnight with 0.5 μg/mL of G. parasuis HS069; recombinant LppA; recombinant LpoA; or recombinant YaeT in 100 mM carbonate-bicarbonate buffer for pigs vaccinated with Combination 1; or coated with 0.5 μg/mL of recombinant catalase; recombinant PBP5; or recombinant LppA in 100 mM carbonate-bicarbonate buffer for pigs vaccinated with Combination 2.

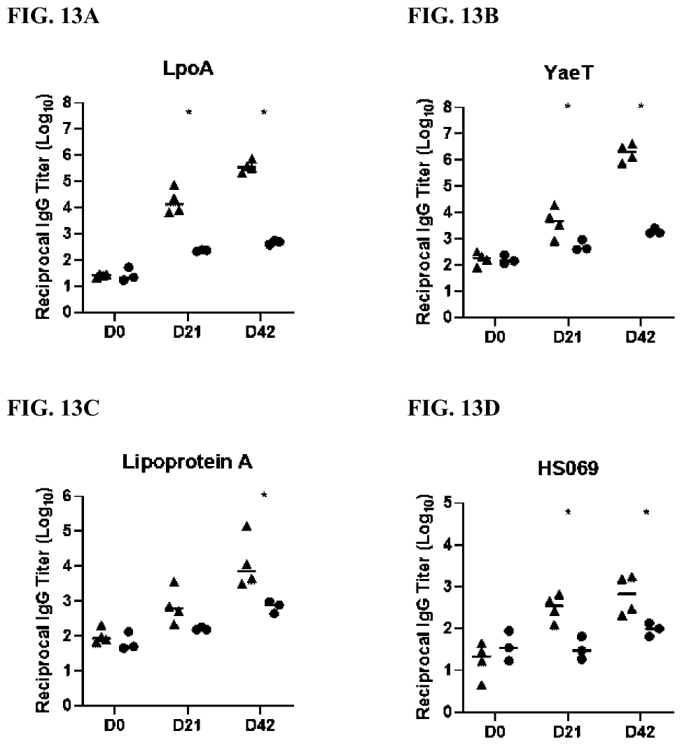

The antibody responses to vaccine antigens from Combination 1 (LppA, LpoA, YaeT) and HS069 after homologous challenge are shown in FIG. 13A to FIG. 13D. As seen in these figures, higher titers were seen in vaccinated animals to the proteins included in the vaccine and the challenge strain (HS069) at the time of challenge (D42). Statistical differences at the P<0.05 level are marked with an asterisk (*).

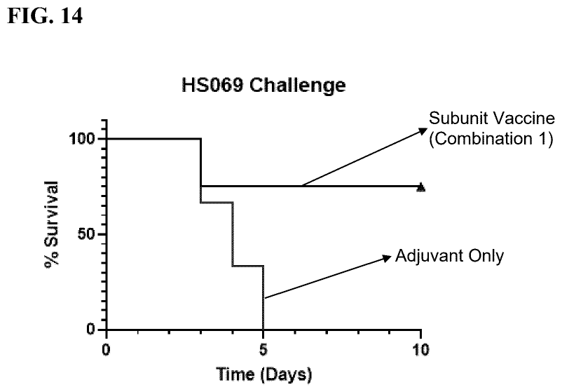

FIG. 14 shows the percent animal survival post challenge with HS069 after vaccination with Combination 1. Animals vaccinated with Combination 1 had better survival (3/4) than control animals (0/3), but statistical significance was not reached due to small study size (P=0.10).

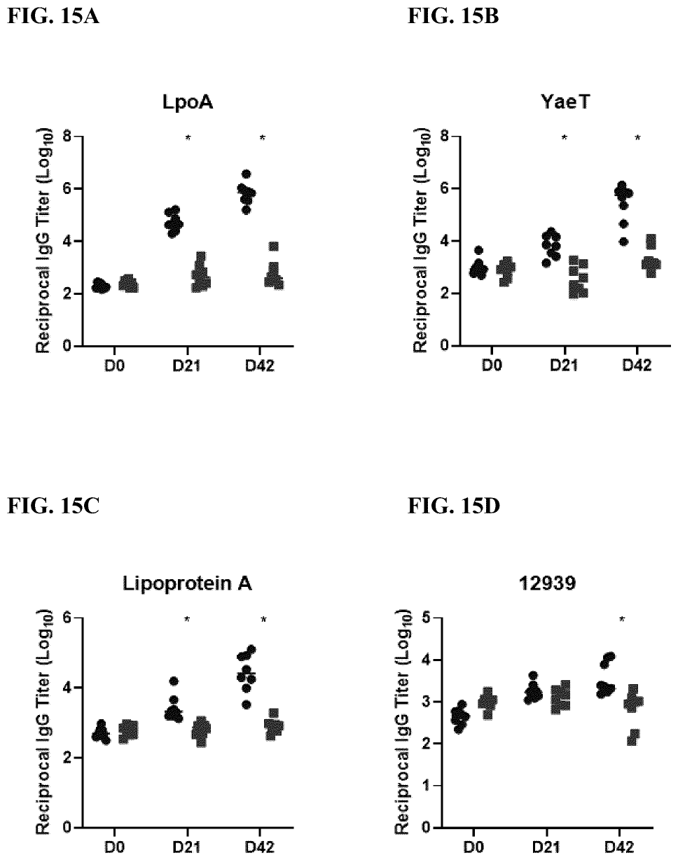

The antibody responses to vaccine antigens from Combination 1 (LppA, LpoA, YaeT) and 12939 after heterologous challenge are shown in FIG. 15A to FIG. 15D. As seen in these figures, higher titers were seen in vaccinated animals to the proteins included in the vaccine and the challenge strain (12939) at the time of challenge (D42). Statistical differences at the P<0.05 level are marked with an asterisk (*).

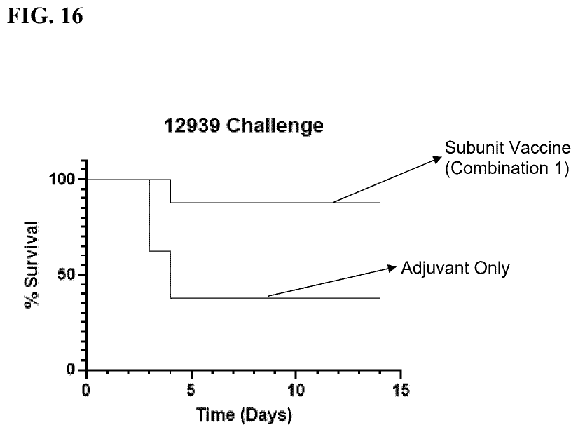

FIG. 16 shows the percent animal survival post challenge with 12939 after vaccination with Combination 1. Animals vaccinated with Combination 1 had statistically improved survival (7/8) compared to control animals (3/8) following challenge (P=0.04).

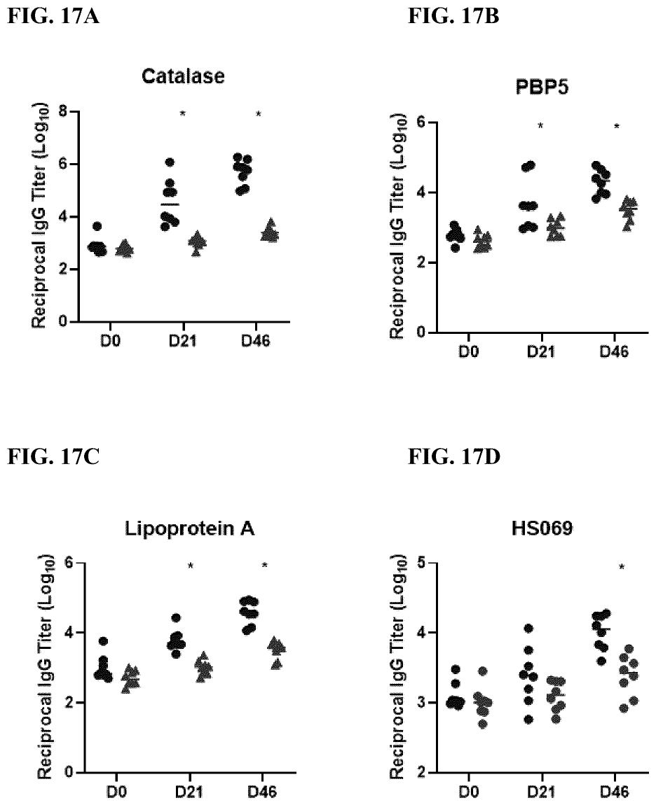

The serum antibody response to vaccine antigens from Combination 2 (catalase, PBP5, LppA) after homologous challenge with HS069 was evaluated by ELISA. The results are shown in FIG. 17A to FIG. 17D. As seen in these figures, Higher titers were seen in vaccinated animals to the proteins included in the vaccine and the challenge strain at the time of challenge (D46). Statistical differences at the P<0.05 level are marked with an asterisk (*).

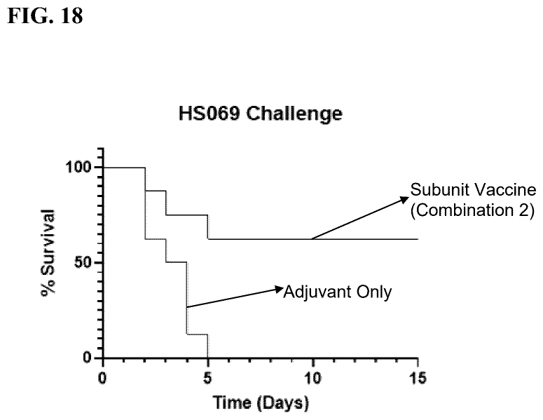

FIG. 18 shows the percent animal survival following heterologous challenge after vaccination with Combination 1. Animals vaccinated with Combination 1 had statistically improved survival (7/8) over control animals (3/8) (P=0.04).

The data in this Example shows that subunit vaccine combinations containing LppA provide protection to vaccinated animals as compared to animals vaccinated with adjuvant only.

### Example 7

### Vaccine Study with rLppA Alone or in Combination

The effect of subunit vaccines comprising a recombinant lipoprotein A (LppA) either alone or employed as a vaccine additive with a bacterin was studied.

Twenty seven cesarean derived, colostrum deprived (CDCD) pigs derived at the National Animal Disease Center were distributed into four groups. Group 1 (n=6) was vaccinated with a vaccine containing 50 μg of Lipoprotein A in PBS with 20% Emulsigen™-D (MVP Laboratories Inc). Group 2 (n=5) was vaccinated with Nagasaki Bacterin (10⁹) with 20% Emulsigen™-D. Group 3 (n=6) was vaccinated with Nagasaki Bacterin (10⁹) plus 50 μg of Lipoprotein A in PBS with 20% Emulsigen™-D. Group 4 (n=5) was vaccinated with PBS with 20% Emulsigen™-D. Animals were vaccinated on DO and D21 and challenged intranasally on D42 with 2 mL of 1×10⁹ CFU/mL G. parasuis 12939. Following challenge, animals were evaluated twice daily for clinical signs of G. parasuis. If clinical signs were present but not severe, a third evaluation was completed approximately 5 hours later. Surviving animals were euthanized 14 days post-challenge. At necropsy, the following samples were collected for bacteriologic evaluation: nasal swab, serosal swab, joint fluid, cerebrospinal fluid (CSF), serum, and lung lavage. Serum was collected on DO, D21, and D42 and stored at −80° C.

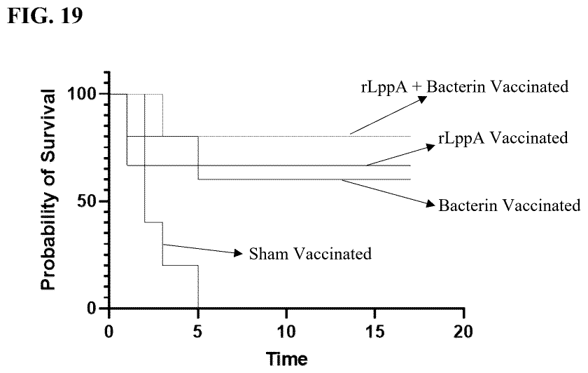

Survival for each group was compared to the adjuvant only group using a Fisher's exact test. As seen in FIG. 19, 4 of 6 animals vaccinated with Lipoprotein A survived (p=0.06); 3 of 5 animals vaccinated with Bacterin survived (p=0.17); 5 of 6 animals vaccinated with Lipoprotein A+Nagasaki Bacterin survived (p=0.002); and 0 of 5 animals vaccinated with adjuvant only survived.

This example shows that Lipoprotein A can protect pigs from G. parasuis disease and it appears to be more effective in protecting pigs from G. parasuis than Nagasaki bacterin alone, and that a mixture of these two provides improved protection.

We claim:

1. A composition for protecting pigs against Glaesserella parasuis infection, the composition comprising at least one recombinant G. parasuis protein selected from Lipoprotein A (LppA), Thiamine biosynthesis protein ApbE, penicillin-binding protein activator LpoA, outer membrane protein assembly protein YaeT, catalase, D-alanyl-D-alanine carboxypeptidase fraction A (PBP5), spermidine/putrescene ABC transporter ATPase protein (PotA), or a combination thereof, and a veterinarily-acceptable adjuvant or carrier.

2. The composition of claim 1, wherein LppA has an amino acid sequence at least 80% homologous to SEQ ID NO: 1 over the length of SEQ ID NO: 1 that is in common with said polypeptide; AbpE has an amino acid sequence at least 80% homologous to SEQ ID NO: 2 over the length of SEQ ID NO: 2 that is in common with said polypeptide; LpoA has an amino acid sequence at least 80% homologous to SEQ ID NO: 3 over the length of SEQ ID NO: 3 that is in common with said polypeptide; YaeT has an amino acid sequence at least 80% homologous to SEQ ID NO: 4 over the length of SEQ ID NO:4 that is in common with said polypeptide; PBP5 has an amino acid sequence at least 80% homologous to SEQ ID NO: 5 over the length of SEQ ID NO: 5 that is in common with said polypeptide; PotA has an amino acid sequence at least 80% homologous to SEQ ID NO: 6 over the length of SEQ ID NO: 6 that is in common with said polypeptide; and catalase has an amino acid sequence at least 80% homologous to SEQ ID NO: 7 over the length of SEQ ID NO: 7 that is in common with said polypeptide.

3. The composition of claim 1, wherein the composition is an antigen polypeptide, a vaccine, or an immunogenic composition.

4. The composition of claim 3, wherein the composition comprises a recombinant LppA.

5. The composition of claim 4, wherein the composition comprises a recombinant LppA and a bacterin vaccine.

6. The composition of claim 1, wherein the composition comprises LppA.

7. The composition of claim 6, wherein the composition further comprises LpoA, and YaeT.

8. The composition of claim 6, wherein the composition further comprises catalase and PBP5.

9. The composition of claim 5, wherein the composition comprises Nagasaki bacterin.

10. The composition of claim 1, wherein the composition comprises ApbE, LpoA, and YaeT.

11. The composition of claim 1, wherein the composition comprises catalase, PBP5, and PotA.

12. A method for provoking an immune response in a pig, the method comprising administering to the pig at least one effective dose of a composition comprising at least one recombinant G. parasuis LppA, ApbE, LpoA, YaeT, catalase, PBP5, PotA, or a combination thereof, and a veterinarily-acceptable adjuvant or carrier.

13. The method of claim 12, wherein LppA has an amino acid sequence at least 80% homologous to SEQ ID NO: 1; AbpE has an amino acid sequence at least 80% homologous to SEQ ID NO: 2; LpoA has an amino acid sequence at least 80% homologous to SEQ ID NO: 3; YaeT has an amino acid sequence at least 80% homologous to SEQ ID NO: 4; PBP5 has an amino acid sequence at least 80% homologous to SEQ ID NO: 5; PotA has an amino acid sequence at least 80% homologous to SEQ ID NO: 6; and catalase has an amino acid sequence at least 80% homologous to SEQ ID NO: 7.

14. The method of claim 12, wherein the method comprises administering a composition comprising LppA.

15. The method of claim 14, wherein the composition further comprises LpoA, and YaeT.

16. The method of claim 14, wherein the composition further comprises catalase and PBP5.

17. The method of claim 14, wherein the composition further comprises Nagasaki bacterin.

18. The method of claim 14, wherein the method comprises administering a composition comprising ApbE, LpoA, and YaeT.

19. The method of claim 14, wherein the method comprises administering a composition comprising catalase, PBP5, and PotA.
